(19) United States
(12) Patent Application Publication
(10) Pub. No.: US 2021/0270703 A1
(43) Pub. Date: Sep. 2, 2021

(54) METHOD FOR ISOLATING NUCLEI AND CELLS FROM TISSUES

(71) Applicant: 10X Genomics, Inc., Pleasanton, CA (US)

(72) Inventor: Jawad N. Abousoud, Pleasanton, CA (US)

(73) Assignee: 10X Genomics, Inc., Pleasanton, CA (US)

(21) Appl. No.: 17/187,096

(22) Filed: Feb. 26, 2021

Related U.S. Application Data
(60) Provisional application No. 62/983,278, filed on Feb. 28, 2020.

(51) Int. Cl. G01N 1/30; G01N 33/483

(52) U.S. Cl. CPC G01N 1/30; G01N 2001/305; G01N 33/4833

(57) ABSTRACT

A method of extracting and isolating fixed biological particles, such as fixed cells and/or fixed nuclei from snap-frozen biological tissue yields dramatically improved amounts of ligation products and an increase in targeted single cell RNA sequence data quality and sensitivity in subsequent RNA-templated ligation. The method preserves the integrity of the biological particles, such as cells and/or or nuclei from biological tissue samples during extraction and isolation by reducing the amount of cell degradation and RNA leakage associated with conventional methods.

### FIELD

This invention relates to methods for isolating fixed biological particles, including fixed cells and/or nuclei from biological tissues, where the biological particles, including cells and/or nuclei, are capable of being successfully used in certain single biological particle (e.g., single-cell or single nucleus) genomics, epigenomics, transcriptomics and proteomics assays.

### BACKGROUND

Recovery of cells and or nuclei from tissues, where the cells can support various biological or biochemical reactions, is an active area of research. In some examples, existing methods may dissociate cells from tissues and subsequently fix the dissociated cells. However, the resulting cells and/or nuclei may not produce good results in single cell assays used for genomic, epigenomic, transcriptomic and proteomic analyses. Attempts at improving conventional cell recovery methods, for example by using rapid (“flash” or “snap”) freezing of biological tissues, in liquid nitrogen for example, prior to dissociation/fixation steps, have not produced satisfactory results.

Methods for producing single cells and/or nuclei from tissues, where the cells/tissues, their genomes, analytes and the like, can be maintained for a time, and then be used in single cell workflows to produce successful results (e.g., comparable to results obtained with fresh cells), would be advantageous to research, development and diagnosis in the single cell analysis field.

### SUMMARY

Disclosed here are methods for obtaining single biological particles (e.g., single cells and/or single nuclei) from tissues, where the single biological particles (e.g., cells/nuclei) are capable of supporting certain biological/biochemical reactions. Generally, the biological particles, including cells and/or nuclei, obtained from the tissues using the methods are fixed. Generally, the disclosed methods may not include specific steps for reversing the fixation process (e.g., using decrosslinking agents, un-fixing agents, or reversible fixation agents). High quality biological particles, such as cells/nuclei, obtained from the disclosed processes, as assessed by results of certain single-cell genomic, epigenomic, transcriptomic and/or proteomic assays, is achieved. In some examples, the disclosed methods produce fixed single biological particles (e.g., single cells and/or single nuclei) having analytes (e.g., mRNA, protein) and/or genomes capable of supporting certain enzymatic reactions. The assays may be single biological particle (e.g., single-cell or single nucleus) assays, including partition-based assays, flow cytometric assays, and the like. In some examples, the recovered single biological particles (e.g., single cells/nuclei) may have nucleic acid molecules of interest (e.g., mRNA molecules) that can be used to in nucleic acid reactions, including templated nucleic acid ligation reactions using nucleic acid probes. In one embodiment, the templated nucleic acid ligation is an RNA-templated ligation reactions (i.e., two DNA probes ligated while hybridized to an RNA template). In some examples, the recovered single biological particles (e.g., single cells/nuclei) may have proteins or other antigens that that can be stained by various methods, including specific staining with antibodies.

In some examples, disclosed is a method for nucleic acid analysis of tissue samples that comprise (a) contacting a tissue sample with a fixing agent; (b) in the presence of the fixing agent, dividing the tissue sample into tissue segments to allow perfusion of the fixing agent into the tissue segments; (c) dissociating the tissue segments to provide a plurality of biological particles, wherein said biological particles comprise a plurality of sample nucleic acid molecules; and (d) generating a plurality of barcoded nucleic acid molecules using said plurality of sample nucleic acid molecules and a plurality of nucleic acid barcode molecules. In some examples, prior to step (d), the method further comprises hybridizing a plurality of nucleic acid probes to the plurality of sample nucleic acid molecules of the plurality of biological particles.

In some examples, a method of extracting and isolating fixed cells and/or nuclei from a biological tissue is disclosed, comprising obtaining a fixed tissue sample; and dissociating the fixed tissue sample to obtain cells and/or nuclei. In some examples, the method further comprises forming a suspension of the cells and/or nuclei and filtering the suspension. In some examples, the method further comprises performing a single-cell assay using the cells and/or nuclei.

In some examples, disclosed is a method of ribonucleic acid (RNA) analysis in biological tissue samples, comprising contacting a biological tissue sample with an organic fixing agent; in the presence of the organic fixing agent, dividing the biological tissue sample into tissue segments to allow perfusion of the organic fixing agent into the tissue segments; dissociating the tissue segments to provide a plurality of single cells and/or single nuclei; and performing RNA analysis on the plurality of single cells and/or single nuclei.

Also disclosed are compositions of cells and/or nucleic obtained by any of the methods disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

The following U.S. patents and U.S. published patent applications are each incorporated by reference in their entirety into this application:

U.S. Pat. No. 9,644,204 (Ser. No. 14/175,935), issued May 9, 2017 and titled, “Partitioning And Processing Of Analytes And Other Species”;

U.S. Pat. No. 9,975,122 (Ser. No. 14/934,044), issued May 22, 2018 and titled, “Instrument Systems For Integrated Sample Processing”;

U.S. Pat. No. 10,053,723 (Ser. No. 15/719,459), issued Aug. 21, 2018 and titled, “Capsule Array Devices And Methods Of Use”; and

U.S. Pat. No. 10,071,377 (Ser. No. 15/687,856), issued Sep. 11, 2018 and titled, “Fluidic Devices, Systems, And Methods For Encapsulating And Partitioning Reagents, And Applications Of Same”.

Other references incorporated by reference may be listed throughout the application.

### BRIEF DESCRIPTION OF THE DRAWINGS

In the accompanying drawings, which are incorporated in and constitute a part of the specification, embodiments of the disclosed inventions are illustrated. It will be appreciated that the embodiments illustrated in the drawings are shown for purposes of illustration and not for limitation. It will be appreciated that changes, modifications and deviations from the embodiments illustrated in the drawings may be made without departing from the spirit and scope of the invention, as disclosed below.

FIG. 1 is a schematic diagram of steps in an example method for producing single fixed cells from tissues.

FIG. 2 shows an example of a microfluidic channel structure for partitioning individual biological particles.

FIG. 3 shows an example of a microfluidic channel structure for delivering barcode carrying beads to droplets.

FIG. 4 shows an example of a microfluidic channel structure for co-partitioning biological particles and reagents.

FIG. 5 shows an example of a microfluidic channel structure for the controlled partitioning of beads into discrete droplets.

FIG. 6 shows an example of a microfluidic channel structure for increased droplet generation throughput.

FIG. 7 shows another example of a microfluidic channel structure for increased droplet generation throughput.

FIG. 8 is a bioanalyzer plot of image intensity versus size in base pairs for fixed dissociated tissue cells and cell nuclei produced according to the invention, following further processing using an RNA-templated ligation protocol.

FIG. 9 is a replicate of the 3000-cell load electropherogram alone with the bp size highlighted for the ligation products at 230 bp.

FIG. 10 is a bioanalyzer plot of image intensity versus size in base pairs for fixed dissociated cell nuclei produced according to a conventional method, following further processing using the RNA-templated ligation protocol.

FIG. 11 is a barcode rank plot of UMI counts versus barcodes for fixed dissociated tissue cells and fixed nuclei produced according to the invention, following further processing using the RNA-templated ligation protocol.

FIG. 12 is a barcode rank plot of UMI counts versus barcodes for dissociated cell nuclei produced according to the conventional method, following further processing using the using the Single Cell 3′ Regent Kit, Version 3, available from 10× Genomics.

FIG. 13 schematically illustrates an example microwell array.

FIG. 14 schematically illustrates an example microwell array workflow for processing nucleic acid molecules.

FIG. 15 depicts an example of a barcode carrying bead.

FIG. 16 schematically illustrates examples of labelling agents.

FIG. 17A and FIG. 17B schematically depict an example workflow for processing nucleic acid.

### DETAILED DESCRIPTION

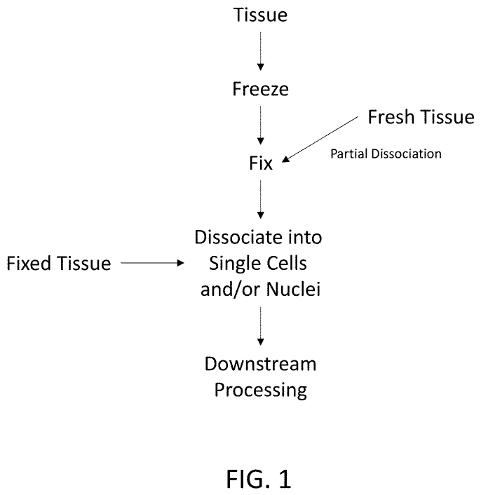

The methods disclosed herein are designed to produce dissociated biological particles (e.g., cells and/or nuclei) from tissue samples, such that the biological particles (e.g., cells/nuclei) are of sufficient quality to produce good results in certain single-cell workflows, including workflows for genomic, epigenomic, transcriptomic and proteomic analyses. In some examples, as shown in FIG. 1, a tissue sample may be obtained and flash frozen. The frozen tissue sample may be fixed. In some examples, fresh tissue may be fixed without the flash-freezing step. Prior to and/or during fixation, the cells of the tissue may be dissociated or partially dissociated from the tissue. In some examples, dissociation/partial dissociation may be performed mechanically. Fixation may be terminated, in some examples by quenching. Dissociation of biological particles (e.g., cells or nuclei) from the tissue, into single biological particles (e.g., single cells and/or nuclei) may be performed, in some examples using enzymes, with or without mechanical dissociation. Biological particles (e.g., cells or nuclei) which have been previously fixed may enter the sequence of steps at this dissociation step. In some examples, dissociated biological particles (e.g., cells/cell clumps) resulting from the dissociation step may be filtered to obtain single biological particles (e.g., cells and/or nuclei).

In some embodiments of the methods, tissue samples used in the method can be fresh or fixed. In some embodiments, tissue can be frozen (e.g., snap or flash frozen) or not frozen (e.g., fresh). In some embodiments, tissue entering the fixation step can be dissociated into biological particles, such as cells and/or nuclei, partially dissociated (e.g., into cells and/or nuclei), or not dissociated into single biological particles (e.g., single cells and/or nuclei). In some embodiments, the dissociation of a tissue sample (e.g., into single biological particles, such as single cells or nuclei) can be performed prior to, during, and/or subsequent to the fixation step. In some embodiments, dissociation of the tissue sample can be performed or not performed during a step to quench fixation. In some embodiments, dissociation of the tissue sample can be performed mechanically. In some embodiments, dissociation of the tissue sample can be performed enzymatically. In some embodiments, mechanical and enzymatic dissociation of the tissue sample may be performed simultaneously.

Generally, enzymatic dissociation will not work during a fixation step, or in presence of a fixative that has not been quenched.

### Definitions

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

Herein, “amplification product” refers to molecules that result from reproduction or copying of another molecule. Generally, the molecules copied or reproduced are nucleic acid molecules, specifically DNA or RNA molecules. In some examples, the molecule reproduced or copied may be used as a template for the produced molecules. In some examples, an analyte captured by the capture domain of an oligonucleotide may be used as a template to produce an amplification product. In some examples, an mRNA captured by the capture domain of an oligonucleotide may be used as a template to produce a cDNA amplification product. Various enzymes (e.g., reverse transcriptase) may be used for this process. The cDNA amplification product may in turn act as a template for amplification that may also be called amplification products. Various enzymes (e.g., Taq polymerase) may be used for this process.

Herein, “analyte” refers to a substance whose chemical constituents are being identified and/or measured. Generally, this application refers to analytes from and/or produced by cells. Any or all molecules or substance from or produced by a cell may be referred to herein as analytes. Chemically, cellular analytes may include proteins, polypeptides, peptides, saccharides, polysaccharides, lipids, nucleic acids, and other biomolecules.

Herein, “barcode,” generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). Barcodes can have a variety of different formats. For example, barcodes can include polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads. In some examples, a barcode may be a nucleotide sequence that is encoded by, linked to or associated with one or more oligonucleotides. In some examples, a specific barcode may correlate with a location of a barcode, on a support, for example. A barcode used to convey locational information may be called a spatial barcode.

Herein, “barcoded molecule” or, in some examples, “barcoded nucleic acid molecule” generally refers to a molecule or a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence (e.g., targeted by a primer sequence) or a non-targeted sequence. For example, in the methods, systems and kits described herein, hybridization and reverse transcription of the nucleic acid molecule (e.g., a messenger RNA (mRNA) molecule) of a cell with a nucleic acid barcode molecule (e.g., a nucleic acid barcode molecule containing a barcode sequence and a nucleic acid primer sequence complementary to a nucleic acid sequence of the mRNA molecule) results in a barcoded nucleic acid molecule that has a sequence corresponding to the nucleic acid sequence of the mRNA and the barcode sequence (or a reverse complement thereof). A barcoded nucleic acid molecule may be a nucleic acid product, such as a nucleic acid ligation product. The ligation product may comprise two probes (e.g., DNA or DNA-containing probes) ligated using an RNA template. Barcoding of the ligation product may occur through a barcode sequence as part of one or both probes or by subsequent appending of a barcode sequence to the ligation product. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the mRNA as well as the sequence of the spatial barcode thereby determining the locational position of the mRNA along with its identity. Herein, molecules stated to have a “common barcode sequence” refers to molecules that are labeled or identified with the same barcode sequence.

Herein, “base-paired” generally refers to the situation where two complementary nucleic acids have formed hydrogen bonds between complementary nucleotides in the different strands. Two such nucleic acid strands may be referred to as hybridized to one another.

Herein, “branched” generally refers to a particular arrangement of oligonucleotide capture probes within an assembly that increases the density of capture domains in a space.

Herein, “bind” generally refers to a stable physical interaction between substances. Cells may bind to other cells. Cells may bind to molecules. Molecules may bind to cells. Molecules may bind to other molecules. In some examples, binding of substances may be specific. Example specific binding events include cell receptor binding of a ligand and antibody binding of an antigen. In some examples, two substances that specifically bind to one another may have a higher affinity for each other than two substances that non-specifically bind to each other or which do not bind to each other. Under certain conditions, specific binding of substances may occur, while non-specific binding of substances may not occur. “Binding” refers to causing substances to bind.

The term “biological particle,” as used herein, generally refers to a discrete biological system derived from a biological sample. The biological particle may be a macromolecule, small molecule, virus, cell, cell derivative, cell nucleus, cell organelle, cell constituent and the like. Examples of a cell organelle include, without limitation, a nucleus, endoplasmic reticulum, a ribosome, a Golgi apparatus, an endoplasmic reticulum, a chloroplast, an endocytic vesicle, an exocytic vesicle, a vacuole, and a lysosome. The biological particle may contain multiple individual components, such as macromolecules, small molecules, viruses, cells, cell derivatives, cell nuclei, cell organelles and cell constituents, including combinations of different of these and other components. The biological particle may be or may include DNA, RNA, organelles, proteins, or any combination thereof. These components may be extracellular. In some examples, the biological particle may be referred to as a clump or aggregate of combinations of components. In some instances, the biological particle may include one or more constituents of a cell but may not include other constituents of the cell. An example of such constituents include nucleus or an organelle. A cell may be a live or viable cell. The live cell may be capable of being cultured, for example, being cultured when enclosed in a gel or polymer matrix or cultured when comprising a gel or polymer matrix. A biological particle may include a single cell and/or a single nuclei from a cell.

Herein, “capable” means having the ability or quality to do something.

Herein, “capture” generally refers to the capability of a first substance to interact with and/or bind a second substance where, for example, the second substance is part of a population of other substances. An analyte may be captured. In some examples, capture refers to identification of a target nucleic acid molecule (e.g., an RNA) by its hybridization to a capture probe, and/or amplification of a target nucleic acid molecule or a nucleic acid probe hybridized to it (e.g., an RNA or a probe hybridized to the RNA) using, for example polymerase chain reaction (PCR) and/or nucleic acid extension of a target nucleic acid molecule or a capture probe hybridized to it using, for example reverse transcription reactions.

Herein, “capture probe” refers to a molecule (e.g., an oligonucleotide) that contains a capture domain.

Herein, “capture domain” means a part of a molecule that is capable of binding or capturing a substance. An analyte capture domain may be capable of capturing analytes that may include proteins, polypeptides, peptides, saccharides, polysaccharides, lipids, nucleic acids, and other biomolecules. In some examples, the analyte capture domain may be a nucleotide sequence capable of hybridizing to an analyte that contains a complementary nucleotide sequence. Herein, “nucleotide capture sequence” refers to a first nucleotide sequence that is capable of capturing (e.g., by hybridizing to) a second nucleotide sequence. In some examples, an analyte capture domain may contain modified nucleotides.

Herein, “cell type” is a characterization used to describe a group or population of cells that have at least one characteristic in common. In some examples, cells from a tissue may be of the same cell type. In some examples, different cell types may be in a tissue.

Herein, “complementary,” in the context of one sequence of nucleic acids being complementary to another sequence, refers to the ability of two strands of single-stranded nucleic acids to form hydrogen bonds between the two strands, along their length. A complementary strand of nucleic acids is generally made using another nucleic acid strand as a template. A first nucleotide that is capable of hybridizing to a second nucleotide sequence may be said to be a complement of the second nucleotide sequence. A first nucleic acid sequence that is complementary to a second nucleic acid sequence may also be referred to as the reverse complement of the second nucleic acid sequence, e.g., a first nucleic acid sequence or reverse complement thereof.

Herein, “configured to” generally refers to a component of a system that can perform a certain function.

Herein, “contact” refers to physical touching of separate substances or objects. “Contacting” refers to causing separate substances to physically touch one another.

Herein, “crosslinking,” means connecting or attaching two or more separate substances to each other. The connecting or attaching is due to formation of crosslinks. In some examples, crosslinking refers to formation of chemical bonds between two or more atoms in a molecule or in different molecules.

Herein, “dissociate” generally refers to a process whereby multiple biological particles (e.g., cells and/or nuclei) that are associated with one another (e.g., contacting one another, as in a tissue sample) are separated such that they not contacting one another or, at least, are not held together in a mass as configured in some tissues. In some examples, dissociated cells and/or nuclei may appear as single cells and/or nuclei in solution and under a coverslip, as viewed in a light microscope. Dissociation may use chemical, enzymatic and/or mechanical methods. A tissue may be, for example, chopped or cut into smaller tissue segments initially and the tissue segments may be dissociated into biological particles (e.g., cells or nuclei).

Herein, “duplex” refers to a double-strand nucleic acid. Herein, duplexes are generally formed between complementary hybridizing nucleotide sequences. The double strand (or double stranded) nucleic acid may comprise two strands having the same or different length. The duplex may be a fully or partially double stranded nucleic acid duplex.

Herein, “enzyme” generally refers to molecules capable of catalyzing biochemical reactions. Herein, enzymes may be used to catalyze biochemical reactions that can cause or contribute to cell dissociation. Such enzymes may be referred to as dissociation enzymes.

Herein, “extract,” is generally used in the context of extracting biological particles (e.g., cells and/or nuclei) from tissues. In that sense, extract may have a meaning similar to dissociating biological particles (e.g., cells and/or nuclei).

Herein, “fresh,” generally in the context of a fresh tissue means that the tissue has recently been obtained from an organism, generally before any processing steps, like flash freezing or fixation.

Herein, “fix,” refers to formation of covalent bonds, such as crosslinks, between biomolecules or within molecules. The process of fixing tissue samples or biological particles (e.g., cells and nuclei) for example, is called “fixation.” The agent that causes fixation is generally referred to as a “fixative” or “fixing agent.” “Fixed biological particles” (e.g., fixed cells or nuclei) or “fixed tissues” refers to biological particles (e.g., cells or nuclei) or tissues that have been in contact with a fixative under conditions sufficient to allow or result in formation of intra- and inter-molecular crosslinks between biomolecules in the biological sample. Fixation may be reversed and the process of reversing fixation may be referred to as “un-fixing” or “decrosslinking.” Unfixing or decrosslinking refers to breaking or reversing the formation of covalent bonds in biomolecules formed by fixatives. Non limiting examples of fixatives or fixing agents include methanol, paraformaldehyde, formalin, and acetone. Other fixing agents may include alcohol, ketone, aldehyde, cross-linking agents, disuccinimidyl suberate (DSS), dimethylsuberimidate (DMS), formalin, dimethyladipimidate (DMA), dithio-bis(-succinimidyl propionate) (DSP), disuccinimidyl tartrate (DST), ethylene glycol bis (succinimidyl succinate) (EGS), bis-imidazole-carboxylate compounds, and combinations thereof.

Herein, “hybridize” refers to a nucleotide sequence of a single-stranded nucleic acid molecule forming a complex with a nucleic acid molecule having a complementary nucleotide sequence. Generally, the complex forms through hydrogen bonding between complementary nucleotide bases in separate nucleic acid molecules.

Herein, “hybridizing nucleotide sequence” refers to a nucleotide sequence, within an oligonucleotide for example, that is capable of hybridizing with a complementary nucleotide sequence in a target nucleic acid molecule present on or within a cell from a tissue sample (e.g., cellular RNA). When a hybridizing nucleotide sequence is of such a length that it hybridizes with a complementary, either fully or partially, nucleotide sequence that is unique to a target nucleic acid molecule(s) (e.g., cellular RNA or family of RNAs), the hybridizing nucleotide sequence may be said to hybridize to the same target nucleic acid molecule (e.g., the same RNA).

Herein, “immobilize” means to restrict or prevent movement.

Herein, “library” refers to a collection of molecules having nucleotide sequences that are generally representative (e.g., comprising the same nucleotide sequences or complementary nucleotide sequences) of nucleotide sequences present in the molecules from the target nucleic acids. Generally, the molecules from which a library is made act as templates for synthesis of the collection of molecules that make up the library. The “library” may be, or may be produced from, amplification products of the target nucleic acid. Herein, libraries can be created from amplification of a mRNA analyte, or copies thereof, captured on an array. Therefore, the library can be derived from the captured target nucleic acid.

Herein, “isolated” means to separate a first substance from one or more other substances such that the first substance is pure, partially pure or in a free state.

Herein, “oligonucleotide” means a linear polymer of nucleotides, in some examples 2′-deoxyribonucleotides. Oligonucleotides are single stranded. Oligonucleotides can be of various lengths. Oligonucleotides can include modified nucleotides as known in the art.

Herein, “partition” generally, refers to a space or volume that may be suitable to contain one or more species or conduct one or more reactions or processes. A partition may be a physical compartment, such as a droplet or well (e.g., a microwell). The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase.

Herein, “perfusion” means to cause or facilitate a liquid to flow into a substance.

Herein, “permeable” refers to something that allows certain materials to pass through it. “Permeable” may be used to describe a biological particle, such as a cell or nucleus, in which analytes in the biological particle can leave the biological particle. “Permeabilize” is an action taken to cause, for example, a biological particle (e.g., a cell) to release its analytes. In some examples, permeabilization of a biological particle is accomplished by affecting the integrity of a biological particle membrane (e.g., a cellular or nuclear membrane) such as by application of a protease or other enzyme capable of disturbing a membrane allowing analytes to diffuse out of the biological particle.

Herein, “primer” means a single-stranded nucleic acid sequence that provides a starting point for DNA synthesis. Generally, a primer has a nucleotide sequence that is complementary to a template, and has an available 3′-hydroxyl group to which a transcriptase or polymerase can add additional nucleotides complementary to corresponding nucleotides in the template, to synthesize a nucleic acid strand in the 3′ to 5′ direction.

Herein, “pulverize” refers to mechanical tissue dissociation.

Herein, “quench” refers to stopping or terminating activity of fixing agents on biological particles (e.g., cells) in a tissue.

Herein, “nucleic acid probe” refers to a nucleic acid oligonucleotide or molecule capable of hybridizing to a nucleic acid template molecule, such as a sample nucleic acid template molecule. The nucleic acid probe may comprise RNA or DNA. The nucleic acid probe may comprise ribonucleotides and/or deoxyribonucleotides. In one embodiment, the nucleic acid probe comprises a terminal ribonucleotide.

Herein, “sample” or “biological sample” generally refers to a collection of cells or to a tissue. Generally, a tissue contains multiple biological particles (e.g., cells and nuclei), often similar biological particles (e.g., cells) that may perform the same or similar functions. The sample may be a cell sample. The sample may be a cell line or cell culture sample. The sample can include one or more cells, or one or more cell aggregates or clusters. The biological sample may be derived from another sample. The sample may be a tissue sample, such as a biopsy, core biopsy, needle aspirate, or fine needle aspirate. Example tissue types in animals may include connective, epithelial, brain, adipose, muscle and nervous tissue. The sample may be a fluid sample, such as a blood sample, urine sample, or saliva sample. The sample may be a skin sample. The sample may be a cheek swab. The sample may be a plasma or serum sample. The sample may be a blood sample. In some examples, a sample may comprise any number of macromolecules, for example, cellular macromolecules or cellular analytes. The present disclosure is not limited to any particular type of tissue.

Herein, “single biological particle”, such as a single cell or a single nucleus generally refers to a biological particle that is not present in an aggregated form or clump. Single biological particles, such as cells and/or nuclei may be the result of dissociating a tissue sample.

Herein, “template” refers to one single-stranded nucleic acid acting as a “template” for synthesis of another complementary single-stranded nucleic acid. For example, RNA can act as a template for synthesis of a complementary DNA strand synthesized using reverse transcriptase. A single-stranded DNA can act as a template for synthesis of a complementary DNA strand, most often by a DNA polymerase. DNA or RNA molecules may also act as a template for a templated ligation reaction using nucleic acid probes.

Herein, “un-fix” or “decrosslink,” refers to breaking or reversing the formation of covalent bonds in biomolecules formed by fixatives. “Un-fixing agents” (or “decrosslinking agents”) refer to the substances that cause the un-fixing. Agents that perform these functions may be called un-fixing or decrosslinking agents. In some examples, un-fixing or decrosslinking may occur when a reversible fixing agent is used, and when the fixation caused by these agents is reversed.

Herein, “un-fixed” refers to the processed condition of a cell, a plurality of cells, a tissue sample or any other biological sample that is characterized by a prior state of fixation followed by a reversal of the prior state of fixation. For instance, an un-fixed cell may also be referred to as a “previously fixed” cell. In one embodiment, an un-fixed cell is characterized by broken or reversed covalent bonds in the biomolecules of the cell(s) or sample, where such covalent bonds were previously formed by treatment with a fixation agent (e.g., paraformaldehyde or PFA).

Generally, the methods disclosed herein do not use decrosslinking or un-fixing agents, and do not concern de-crosslinked or un-fixed cells, nuclei, or biological particles.

Herein, “unique molecular identifier” or “UMI” generally refers to an identifier of a particular analyte captured by a capture probe, or a particular ligation product as described herein. In one embodiment, a nucleic acid probe of a pair of nucleic acid probes that can hybridize to a sample nucleic acid molecule of interest may comprise a UMI. Alternatively, a nucleic acid complex as described herein may comprise a UMI, such as by appending following a ligation reaction. In addition, a UMI may be appended to a nucleic acid complex within a partition via the use of partition-specific nucleic acid barcode molecules (e.g., a partition comprising a fixed biological particle and a support comprising nucleic acid barcode molecules, which may comprise UMIs).

### Tissues

Generally, tissue refers to an organization of cells in a structure, where the structure generally functions as a unit in an organism (e.g., mammals) and may carry out specific functions. In some examples, cells in a tissue are configured in a mass and may not be free from one another. This disclosure describes methods of obtaining single biological particles (e.g., cells or nuclei) from tissues that can be used in various single biological particles (e.g., single-cell/nucleus) workflows. In some examples, blood cells (e.g., lymphocytes) can be considered a tissue. However, blood cells, like lymphocytes, generally are free from one another in the blood. The methods disclosed herein can be used to process those cells to obtain cells and/or nuclei, although dissociation steps may not be necessary when using those types of tissues.

Generally, any type of tissue can be used in the methods described herein. Examples of tissues that may be used in the disclosed methods include, but are not limited to connective, epithelial, muscle and nervous tissue. In some examples, the tissues are from mammals.

Tissues that contain any type of cells may be used. For example, tissues from abdomen, bladder, brain, esophagus, heart, intestine, kidney, liver, lung, lymph node, olfactory bulb, ovary, pancreas, skin, spleen, stomach, testicle, and the like. The tissue may be normal or tumor tissue (e.g., malignant). This listing is not meant to be limiting. Although the conditions used in the disclosed may not be identical for different types of tissue, the methods may be applied to any tissue.

The tissues used in the disclosed methods may be in various states. In some examples, the tissues used in the disclosed methods may be fresh, frozen or fixed.

### Tissue Freezinq

In some examples, tissue used in the disclosed methods may be frozen. In some examples, tissue used in the disclosed methods may be fresh tissue that is frozen.

Different methods for freezing tissues may be used. In some examples, the methods used may be rapid methods (e.g., “flash freezing” or “snap freezing”). In some examples, tissues may be lowered to temperatures below about −70° C. using these methods. In some examples, rapid freezing may use ultracold media. In some examples, an ultracold medium may be liquid nitrogen. In some examples, this type of freezing may preserve tissue integrity, in part by preventing the formation of ice crystals that would affect the tissue morphology. In some examples, an ultracold medium may be dry ice.

### Tissue Fixation

Tissue may be fixed using one or more fixing agents. In some examples, the tissue fixed is fresh tissue. In some examples, the tissue fixed may be frozen tissue. In some examples, the tissue fixed may not be dissociated. In some examples, the tissue fixed may be dissociated or partially dissociated (e.g., chopped, cut). In some examples, tissue that has been rapidly frozen and, perhaps, cut or chopped into pieces (e.g., small enough to fit into a tube or container used for fixation) may be used. In some examples, tissue may be dissociated or partially dissociated (e.g., cut, chopped) before or during fixation. In some examples, tissue that is fixed may not be dissociated.

The frozen biological tissue can be fixed using a fixing agent, which is suitably an organic fixing agent. Suitable organic fixing agents include without limitation alcohols, ketones, aldehydes (e.g., glutaraldehyde), cross-linking agents, disuccinimidyl suberate (DSS), dimethylsuberimidate (DMS), formalin, dimethyladipimidate (DMA), dithio-bis(-succinimidyl propionate) (DSP), disuccinimidyl tartrate (DST), ethylene glycol bis (succinimidyl succinate) (EGS), bis(sulfosuccinimidyl)suberate (BS3) and combinations thereof. A particularly suitable fixing agent is a formaldehyde-based fixing agent such as formalin, which is a mixture of formaldehyde and water. The formalin may include about 1% to about 15% by weight formaldehyde and about 85% to about 99% by weight water, suitable about 2% to about 8% by weight formaldehyde and about 92% to about 98% by weight water, or about 4% by weight formaldehyde and about 96% by weight water. In some examples, tissues may be fixed in 4% paraformaldehyde.

Other suitable fixing agents will be appreciated by those of ordinary skill in the art (e.g., International PCT App. No. PCT/US2020/066705, which is incorporated herein by reference in its entirety).

The fixing agent can be chilled and can be at a temperature of about zero to about 100° C., suitably about zero to about 50° C., or about 1 to about 50° C. The fixing agent can be chilled by placing it over a bed of ice to maintain its temperature as close to 0° C. as possible. The frozen biological tissue can be treated with the fixing agent using any suitable technique, suitably by immersing it in the fixing agent for a period of time. Depending on the type and size of the biological tissue sample, the treatment time can range from about 5 minutes to about 60 minutes, suitably about 10 minutes to about 30 minutes, or about 15 minutes to about 25 minutes, or about 20 minutes. In some examples, treatment time may be overnight. During fixing, the snap-frozen tissue will thaw but will suitably remain at a low temperature due to the low temperature environment of the fixing agent.

In some examples, the type/identity of a fixation agent, the amount/concentration of a fixation agent, the temperature at which it is used, the duration for which it is used, and the like, may be empirically determined or titrated. These parameters, and others, may need to be varied to obtain optimal results for different tissues, for different organisms, or for different days on which an experiment is performed. In some examples, insufficient fixation (e.g., too little fixing agent, too low temperature, too short duration) may not, for example, stabilize/preserve the cells/organelles/analytes of tissues. In some examples, excess fixation (e.g., much fixing agent, too high temperature, too long duration) may result in the single biological particles (e.g., cells/nuclei) obtained from the methods not yielding good results in single biological particle (e.g., single-cell or single nucleus) workflows or assays in which the biological particles (e.g., cells or nuclei) are used. Generally, the quality of data obtained in these workflows/assays may be a good measure of the extent of the fixation process.

Generally, the methods disclosed herein do not use decrosslinking or un-fixing agents, and do not concern de-crosslinked or un-fixed cells, nuclei, or biological particles.

During the fixing, the biological tissue sample can be periodically cut into successively smaller segments while it is submerged in the fixation solution, to facilitate perfusion and fixation of the biological tissue sample by the organic fixing agent. For example, the tissue sample may have an initial length, width and/or diameter of about 0.25 cm to about 1.5 cm or may be initially cut into segments having such suitable dimensions. After a first periodic interval, the tissue sample or segments can be cut into smaller segments, and the smaller segments can remain immersed in the fixing agent. This process can be repeated after a second periodic interval, after a third periodic interval, after a fourth periodic interval, and so on. The periodic intervals can range from about 1 to about 10 minutes, or about 2 to about 8 minutes, or about 4 to about 6 minutes. The sum of the periodic intervals can equal the entire fixing time and can range from about 5 to about 60 minutes, or about 10 to about 30 minutes, or about 15 to about 25 minutes, for example. The resulting fixed tissue segments can have a length, width and/or diameter in a range of less than 1 mm to about 10 mm, by way of example.

In some examples, however, the tissue is not cut into smaller segments during fixation. In some examples, this may be performed prior to fixation. In some examples, this may be performed after fixation.

### Quenching the Fixation

Once the biological tissue segments have been sufficiently fixed, the fixation process may be stopped and/or the tissue may be removed from the fixation and the tissue may be washed. Generally, fixation is stopped to cease additional activity of the fixative on the tissue. Fixation may also be stopped so that any subsequent biochemical reactions performed on the tissue (e.g., enzymatic cell dissociation) can function.

In some examples, the tissue segments may be treated or contacted with a quenching medium to quench the fixation. The term “quenching” means to stop the fixation reaction, i.e., the chemical interactions that cause the fixation. Quenching the fixation can be accomplished by immersing the fixed tissue segments in a suitable quenching medium. The fixation quenching medium can be chilled and can have a temperature of about zero to about 100° C., or about 1 to about 50° C.

One suitable quenching medium is a phosphate buffer solution (PBS). One suitable phosphate buffer solution is 1×PBS, available from Sigma Aldrich Corp. 1×PBS has a pH of about 7.4 and the following composition in water: NaCl—137 mM, KCl—2.7 mM, Na₂HPO₄—10 mM, KH₂PO₄—1.8 mM. In one embodiment, the phosphate buffer solution can be combined with fetal bovine serum (FBS) to aid in quenching the fixation reaction. FBS is the liquid fraction of clotted blood from fetal calves, depleted of cells, fibrin and clotting factors, but containing many nutritional and macromolecular factors essential for cell growth. Bovine serum albumin is the major component of FBS. The fetal bovine serum can be combined with the phosphate buffer solution at a concentration of about 1% to about 25% by weight FBS and about 75% to about 99% by weight PBS, suitably about 5% to about 15% by weight FBS and about 85% to about 95% by weight PBS, or about 10% by weight FBS and about 90% by weight PBS. In still another embodiment, a solution of concentrated ethanol in water can be used instead of the PBS in the quenching medium. The ethanol solution can contain about 50% to about 90% by weight ethanol, or about 55% to about 85% by weight ethanol, or about 60% to about 80% by weight ethanol, or about 70% by weight ethanol.

In some examples, fixation may be quenched using a quenching solution that does not contain serum. In some examples, Tris-based buffers may be used. In some examples, PBS+50 mM Tris pH 8.0+0.02% BSA (RNAse free)+0.1 U/ul of RNAse Inhibitor may be used. In some examples, the tissue may be removed from the fixative and washed using a quenching solution or biological buffer.

### Dissociation of the Biological Tissue Segments

The fixed biological tissue segments can be dissociated using a combination of enzymatic or chemical dissociation treatment and pulverization (e.g., mechanical dissociation). The different types of dissociation methods may be used separately, consecutively or simultaneously.

In some examples, mechanical dissociation may include blending, chopping, cutting, mashing, mixing, pestle homogenization, trituration, and other methods.

In some examples, enzymatic dissociation treatment can be performed by treating the fixed biological tissue segments with a dissociation enzyme mixture. The dissociation enzyme mixture can include an enzyme such as papain, dispase, collagenase, hyaluronidase, deoxyribonuclease, ribonuclease, trypsin, chymotrypsin, catalase, elastase, protease, lysozyme and the like can be used for dissociation, either alone or in combination. Suitable enzymes include without limitation Liberase™ available from Sigma Aldrich Corp., which is a lyophilized blend of Collagenase I and II having a pH of about 7.4. In some examples, Liberase™ may be used in combination with one or more other enzymes.

The enzyme(s) can be used in combination with a growth medium that helps to stabilize and preserve the biological particles (e.g., cells and cell nuclei) during dissociation. One suitable growth medium is Roswell Park Memorial Institute (RPMI 1640), available from Thermo Fisher Scientific Corp. and various other sources. RPMI 1640 contains the following ingredients, per liter:

a. Glucose (2 g)

b. pH indicator, phenol red (5 mg)

c. Salts (6 g sodium chloride, 2 g sodium bicarbonate, 1.512 g disodium phosphate, 400 mg potassium chloride, 100 mg magnesium sulfate, and 100 mg calcium nitrate)

d. Amino acids (300 mg glutamine; 200 mg arginine; 50 mg each asparagine, cystine, leucine, and isoleucine; 40 mg lysine hydrochloride; 30 mg serine; 20 mg each aspartic acid, glutamic acid, hydroxyproline, proline, threonine, tyrosine, and valine; 15 mg each histidine, methionine, and phenylalanine; 10 mg glycine; 5 mg tryptophan; and 1 mg reduced glutathione)

e. Vitamins (35 mg i-inositol; 3 mg choline chloride; 1 mg each para-aminobenzoic acid, folic acid, nicotinamide, pyridoxine hydrochloride, and thiamine hydrochloride; 0.25 mg calcium pantothenate; 0.2 mg each biotin and riboflavin; and 0.005 mg cyanocobalamin).

When the enzyme is combined with an enzyme growth medium, the combination may include about 4% to about 96% by weight of the enzyme and about 4% to about 96% by weight of the growth medium, suitably about 6% to about 50% by weight of the enzyme and about 50% to about 94% by weight of the growth medium, or about 8% to about 20% by weight of the enzyme and about 80% to about 92% by weight of the growth medium, or about 10% by weight of the enzyme and about 90% by weight of the growth medium. In one embodiment, the combination includes about 0.1 mg/ml Liberase in RPMI.

The enzyme can be mixed with an enzyme stabilizer. Suitable enzyme stabilizers include without limitation dithiothreitol (DTT), trehalose, Ny-acetyldiaminobutyrate (NADA), ectoine, hydroxyecotine, potassium diaminobutyrate, Tris(2-carboxyethyl)phosphine represented by the formula P(CH2CH2COOH)3 (TCEP), and combinations thereof. Enzyme stabilizers can be combined with the enzyme(s) and present in the enzyme mixture in a concentration of about 2 mM to about 25 mM, suitably about 5 mM to about 15 mM, or about 10 mM. One particularly suitable enzyme stabilizer is DTT.

The enzyme can be mixed with a ribonuclease inhibitor. Small amounts of ribonucleases (RNases) sometimes co-purify with isolated RNA and compromise downstream processing. Such contamination can also be introduced via tips, tubes, and other reagents used in procedures. RNase inhibitors are cytosolic proteins used to inhibit and control such contaminants. The ribonuclease inhibitor can be present in the enzyme mixture in an amount of about 0.05 to about 1.0 units/μl, or about 0.1 to about 0.5 units/μl, or about 0.2 units/μl.

The biological tissue segments can be incubated in the dissociation enzyme mixture at a temperature of about 250° C. to about 450° C., suitably about 32° C. to about 42° C., or about 37° C., for a period of about 3 min to about 30 min, or about 5 min to about 15 min, or about 10 min, and with intermittent titration. Following the dissociation treatment, the tissue segments are subjected to pulverization to form dissociated tissue particles. Pulverization can be accomplished by pushing the tissue segments through a small-pore strainer using a piston, plunger or other suitable forcing device. The small pore strainer can have a median pore size of about 1 micron to about 1000 microns, suitably about 5 microns to about 200 microns, or about 50 microns to about 100 microns. One suitable strainer is a Miltenyi® MACs strainer with a median pore size of about 70 microns. Pulverization can alternatively be accomplished via douncing (e.g., using Dounce homogenizer) or other suitable mechanical means.

In some examples, the amount/concentration of enzymes, the temperature at which the enzymes are used, the duration for which they are used, and the like, may be empirically determined or titrated. In some examples, excess enzyme digestion may affect the ability to obtain suitable cells and/or nuclei from the disclosed methods.

The tissue segments can be forced through the strainer such that the dissociated tissue particles are collected in a tube or other suitable container. The strainer can then be rinsed with a buffering solution and the effluent can be combined with the collected dissociated tissue particles to yield a mixture or suspension. The buffering solution can be a phosphate buffer solution (PBS), and can be the above-described 1×PBS having a pH of about 7.4 and the following composition in water: NaCl—137 mM, KCl—2.7 mM, Na₂HPO₄—10 mM, KH₂PO₄—1.8 mM. Other suitable buffering solutions can also be used. The buffering solution can be combined with a cell nutrient such as bovine serum albumin (BSA), which functions both as a proteinaceous cell nutrient and helps to stabilize the enzymes. The proteinaceous cell nutrient (e.g., BSA) can be added to the buffering solution at any suitable concentration, for example, about 0.01 to about 1% by weight, or about 0.02 to about 0.5% by weight, or about 0.03 to about 0.1% by weight, or about 0.04% by weight. The buffering solution can be chilled, and can have a temperature of about 0° C. to about 10° C., or about 1 to about 5° C. The amount of buffering solution should be enough to enable subsequent centrifugation of the dissociated tissue particles. For example, the amount of buffering solution should be at least about three times the weight of the dissociated tissue particles, or at least about five times the weight of the dissociated tissue particles, or at least about seven times the weight of the dissociated tissue particles, or at least about 10 times the weight of the dissociated tissue particles. The resulting mixture of dissociated tissue particles in buffering solution is ready for centrifuging.

In some examples, depending on the tissue, enzymes may not be used to dissociate cells from tissues. For example, mechanical methods (e.g., cutting, chopping using scissors, razor blades, etc.) may be enough.

Generally, the type of dissociation (e.g., mechanical, enzymatic, mechanical and enzymatic) that yields good results may vary depending on the tissue used (e.g., its size, shape, thickness, fibrosity), how the tissue was processed (e.g., fresh, frozen, extent of fixation), and the like. Generally, for enzymatic dissociation, the specific enzymes and/or combinations of enzymes may vary depending on the tissue used.

### Centrifuging and Filtering

The suspension of dissociated tissue particles and buffering solution can be centrifuged at suitable multiples of gravitational force (g-force) to separate the relatively intact dissociated cell and cell nuclei from fragmented cells and nuclei and other impurities. The centrifuging can be performed at a force of about 50 g to about 2500 g, or about 100 g to about 1500 g, or about 200 g to about 1000 g, or about 300 g to about 700 g, or about 500 g. The centrifuging can be performed while maintaining the temperature of the suspension at about 0° C. to about 10° C., or about 1° to about 5° C. The centrifuging can be performed for a time period long enough to enrich for and/or separate the higher quality, relatively intact dissociated biological particles (e.g., cells and/or cell nuclei) from the lighter weight damaged dissociated material and other impurities. For example, the centrifuging can be performed for about 1 minute to about 15 minutes, or about 2 minutes to about 10 minutes, or about 3 minutes to about 7 minutes, or about 5 minutes. The centrifuging can be performed using any suitable centrifuge apparatus and yields one or more pellets of agglomerated dissociated biological particles (e.g., cells and cell nuclei).

The one or more pellets can then be resuspended in a buffering solution to form a suspension. The buffering solution can be a phosphate buffer solution (PBS), and can be the above-described 1×PBS having a pH of about 7.4 and the following composition in water: NaCl—137 mM, KCl—2.7 mM, Na₂HPO₄—10 mM, KH₂PO₄—1.8 mM. Other suitable buffering solutions can also be used. The buffering solution can be combined with a cell nutrient such as bovine serum albumin (BSA). The proteinaceous cell nutrient (e.g., BSA) can be added to the buffering solution at any suitable concentration, for example, about 0.01 to about 1% by weight, or about 0.02 to about 0.5% by weight, or about 0.03 to about 0.1% by weight, or about 0.04% by weight. The buffering solution can be chilled, and can have a temperature of about zero to about 100 C, or about 10 to about 50 C. The amount of buffering solution should be enough to enable washing followed by subsequent filtration of the dissociated biological particles of the tissue from the suspension. For example, the amount of buffering solution can be at least about three times the weight of the dissociated biological particles, or at least about five times the weight of the dissociated tissue particles, or at least about seven times the weight of the dissociated biological particles, or at least about 10 times the weight of the dissociated biological particles. The resulting suspension of dissociated biological particles in buffering solution is ready for filtration.

The suspension can be filtered to separate the dissociated biological particles (e.g., cells and cell nuclei) from any remaining impurities, yielding a collection or plurality of dissociated biological particles (e.g., cells and cell nuclei). The suspension can be passed to a filter having a pore size of about 1 micron to about 500 microns, or about 5 microns to about 200 microns, or about 10 microns to about 100 microns, or about 25 microns to about 75 microns, or about 40 microns. In some examples, filter size can be from 5 μM to 70 μM. One exemplary filter is a FlowMi® filter available from Sigma Aldrich Corp., and having pore sizes of about 40 microns. The filtration separates the dissociated biological particles (e.g., cells and cell nuclei) from the impurities. In various embodiments, the suspension, centrifugation and filtration can be repeated as many times as are desired to obtain the purest combination of dissociated biological particles (e.g., cells and cell nuclei).

The resulting dissociated biological particles (e.g., cells and cell nuclei) can include, for example, mostly dissociated cells, mostly dissociated cell nuclei, or any combination of dissociated cells and cell nuclei. In one embodiment, depending on the settings of process variables, the dissociated material can range from about 100% dissociated cells to about 100% dissociated cell nuclei and can include any combination in between.

In some examples, biological particles (e.g., cells and/or nuclei) recovered from the disclosed methods, or from individual steps of the disclosed methods, can be visualized using a light microscope, particle analyzer, flow cytometer, and the like. In some examples, these methods may be used to examine and determine optimal parameters of individual steps in the disclosed methods. In some examples, the biological particles (e.g., cells and/or nuclei) recovered may be examined based on how they perform in various single-cell workflows and/or assays.

### Single Biological Particle Assays Using Recovered Cells and Nuclei

The present invention provides methods for further analysis of single biological particles (e.g., single cells and/or single nuclei) obtained by the methods described herein. The recovered biological particles (e.g., cells and/or nuclei) are generally able to support certain single biological particle-assays (e.g., single-cell or nucleus assays) related to, for example, one or more of genomics, epigenomics, transcriptomics, proteomics, and the like. A variety of single-cell biological/biochemical assays may be supported.

In some examples, the recovered cells and/or nuclei disclosed herein may contain mRNA that can act as template for RNA-templated DNA ligation reactions (RTL, e.g., as in Mats Nilsson, Dan-Oscar Antson, Gisela Barbany, Ulf Landegren, RNA-templated DNA ligation for transcript analysis, Nucleic Acids Research, Volume 29, Issue 2, 15 Jan. 2001, Pages 578-581). In some examples, ligated DNA that results from these assays may be a proxy for mRNA and may be used to analyze gene expression on a single-cell basis. In some examples, DNA that results from single cells produced by the disclosed methods may be processed through droplet-based methods, as described in the next section of this application.

In some examples, RTL reactions using biological particles (e.g., cells/nuclei) obtained from the disclosed methods, and ligation probes complementary to regions of a nucleic acid molecule in a biological particle (e.g., an mRNA), produced large amounts of ligation product of the expected size. Single-cell sequencing of the ligation products demonstrated a high number of biological particles (e.g., cells/nuclei) producing expected reads, and a high number of the expected reads per biological particles (e.g., cell/nucleus).

In some examples, the recovered biological particles (e.g., cells and/or nuclei) disclosed herein may be contacted with certain detection molecules, like antibodies that specifically bind certain analytes in the cells and/or nuclei. In some examples, biological particles (e.g., cells/nuclei) stained with, for example, fluorescent antibodies, may be used to detect and quantify antibody-bound antigens. In some examples, the antibody-bound antigens in the biological particles (e.g., cells/nuclei) may be detected and quantified using analytical cytology instruments (Jacobberger, J. W., Fogleman, D. and Lehman, J. M. (1986), Analysis of intracellular antigens by flow cytometry. Cytometry, 7: 356-364).

Processing and Analysis of Dissociated Single Cells and/or Single Nuclei

Some single-cell assays, as described in the previous section, may use droplet-based partitioning in performance of the assays.

Droplet-based (and other partition-based approaches) genomic assays typically involve a biological sample isolated and partitioned as single cells and/or single nuclei in discrete droplets in an emulsion. The discrete droplet usually also includes a unique identifier for the sample in the form of a unique oligonucleotide sequence also contained in the discrete droplet. The discrete droplet can also contain the assay reagents that are used to generate detectable analytes (e.g., 3′ cDNA sequences) from the sample and provide useful genomic information about it (e.g., RNA transcript profile). Further details of methods and compositions for carrying out droplet-based assays are provided elsewhere herein. In one embodiment, the present invention provides a discrete droplet containing a single cell or single nucleus (prepared by the methods described herein) which comprises a plurality of ligation products.

The term “partition,” as used herein, generally, refers to a space or volume that may be suitable to contain one or more species or conduct one or more reactions. A partition may be a physical compartment, such as a droplet or well (e.g., a microwell). The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

One or more reagents may be co-partitioned with a biological particle (e.g., cell or nucleus). For example, a biological particle may be co-partitioned with one or more reagents selected from the group consisting of lysis agents or buffers, permeabilizing agents, enzymes (e.g., enzymes capable of digesting one or more RNA molecules, extending one or more nucleic acid molecules, reverse transcribing an RNA molecule, permeabilizing or lysing a cell, or carrying out other actions), fluorophores, oligonucleotides, primers, probes, barcodes, nucleic acid barcode molecules (e.g., nucleic acid barcode molecules comprising one or more barcode sequences), buffers, deoxynucleotide triphosphates, detergents, reducing agents, chelating agents, oxidizing agents, nanoparticles, beads, and antibodies. In some cases, a biological particle may be co-partitioned with one or more reagents selected from the group consisting of temperature-sensitive enzymes, pH-sensitive enzymes, light-sensitive enzymes, reverse transcriptases, proteases, Iigase, polymerases, restriction enzymes, nucleases, protease inhibitors, exonucleases, and nuclease inhibitors.

Preparation of the partition containing a biological particle (e.g., a cell or nucleus) from a sample that is useful in a partition-droplet-based genomic assay involves numerous steps (e.g., sample transport, tissue dissociation, liquid phase washing and transfer, library preparation) that typically take from a few hours to days. During this preparation time a fresh (i.e., un-fixed) biological sample will begin to degrade and decompose resulting in significant loss of genomic information and assay results that do not reflect the natural state of the sample. The present invention provides compositions and methods for preparing a fixed biological sample that maintain its integrity from the biological point of collection, but which fixed biological sample is capable of being dissociated into single biological particles (e.g., single cells and/or single nuclei), processed for nucleic acid analysis in bulk (e.g., RNA analysis), and then encapsulated (e.g., in a discrete droplet) for a partition-based assay (e.g., generation of a library for sequencing analysis).

As provided in greater detail elsewhere herein, in at least one embodiment, the composition comprises a fixed single biological particle (e.g., a fixed single cell or fixed single nucleus) that has been partitioned (e.g., encapsulated in a discrete droplet), wherein the fixed single biological particle (e.g., a single cell or single nucleus) comprises a plurality of nucleic acid products (e.g., probe-bound nucleic acid molecules and/or nucleic acid ligation products). In one embodiment, the plurality of nucleic acid products is a plurality of nucleic acid templated ligation products (e.g., RNA templated ligation products). In at least one embodiment, the method for preparing such a composition comprises: providing a plurality of fixed single biological particles (e.g., single cells or single nuclei) in bulk and contacting them with pairs of nucleic acid probes that target a plurality of sample nucleic acid molecules (e.g., sample RNA sequences), and allowing the pairs of nucleic acid probes to hybridize to the plurality of sample nucleic acid molecules (e.g., sample RNA sequences), thereby forming a plurality of nucleic acid complexes. In one embodiment, a nucleic acid complex of the plurality of nucleic acid complexes comprises a first probe, a second probe, and a sample nucleic acid molecule. In another embodiment, the first probe can hybridize (or hybridizes) to a first region of the sample nucleic acid molecule and the second probe can hybridize (or hybridizes) to a first region of the sample nucleic acid molecule, wherein the first region and said second region are adjacent to one another. In another embodiment, the first region and the second region are present on a contiguous nucleic acid molecule.

In another embodiment, the method further comprises ligating the hybridized probes (i.e., the first probe to the first region and the second probe to the second region) that are part of the nucleic acid complexes to provide a plurality of fixed biological particles (e.g., single cells or single nuclei) comprising templated ligation products. In one embodiment, a fixed single biological particle (e.g., a fixed single cell or fixed single nucleus) comprises a plurality of templated ligation products, which comprise the first probe ligated to the second probe on the sample nucleic acid molecule. In one embodiment, templated ligation products RNA templated ligation products.

In another embodiment, the present disclosure provides an assay method, wherein the method comprises (a) generating a discrete partition (e.g., droplet or microwell) comprising a fixed biological particle (e.g., a fixed single cell or fixed single nucleus) comprising the nucleic acid products, e.g., ligation products, such as templated ligation products, including RNA templated ligation products (as described herein) and assay reagents for processing the nucleic acid products; and (b) detecting nucleic acid products (e.g., ligation products) from the reaction of the assay reagents and the nucleic acid products.

Those of ordinary skill will appreciate other suitable methods for nucleic acid (e.g., RNA) templated ligation (see e.g., U.S. Publication No. 20200239874A1, WO/2019/157529, and WO/2019/165318, each of which is incorporated by reference in its entirety).

The compositions and methods of the present disclosure can allow for the use of a wide range of biological samples in single-cell droplet-based assays. The term “biological sample,” as used herein refers to any sample of biological origin that includes a biomolecule, such as a nucleic acid, a protein, a carbohydrate, and/or a lipid. Biological samples used in the methods and compositions of the disclosure include blood and other liquid samples of biological origin, solid tissue samples such as a tissue sample (i.e., tissue specimen), a biopsy (i.e., a biopsy specimen), or tissue cultures or cells derived therefrom and the progeny thereof. This includes samples that have been manipulated in any way after isolation from the biological source, such as freezing; washed; or enriched for certain cell populations, such as cancer cells, neurons, stem cells, etc. The term also encompasses samples that have been enriched for particular types of molecules, e.g., nucleic acids, polypeptides, etc. “Biological sample” encompasses a clinical sample, and also includes tissue obtained by surgical resection, tissue obtained by biopsy, cells in culture, cell supernatants, cell lysates, tissue samples (i.e., tissue specimens), organs, bone marrow, blood, plasma, serum, and the like. A “biological sample” also includes a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample having cells (e.g., cancer cells) from a patient.

It is contemplated that the biological samples used in the compositions and methods of the present disclosure can be derived from another sample. Biological samples can include a tissue sample, such as a biopsy, core biopsy, needle aspirate, or fine needle aspirate. Biological samples also include a biological fluid sample, such as a blood sample, urine sample, or saliva sample, or the biological sample may be a skin sample, a cheek swab. The biological sample may be a plasma or serum sample. The biological sample may include cells or be a cell-free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

Methods, techniques, and protocols useful for partitioning biological particles (e.g., individual cells or nuclei, biomolecular contents of cells, etc.) from samples into discrete droplets are described in the art. The discrete droplets generated act a nanoliter-scale container that can maintain separation the droplet contents from the contents of other droplets in the emulsion. Methods and systems for creating stable discrete droplets encapsulating individual biological particles (e.g., cells or nuclei) from biological samples in non-aqueous or oil emulsions are described in, e.g., U.S. Patent Application Publication Nos. 2010/0105112 and 2019/0100632, each of which is entirely incorporated herein by reference for all purposes.

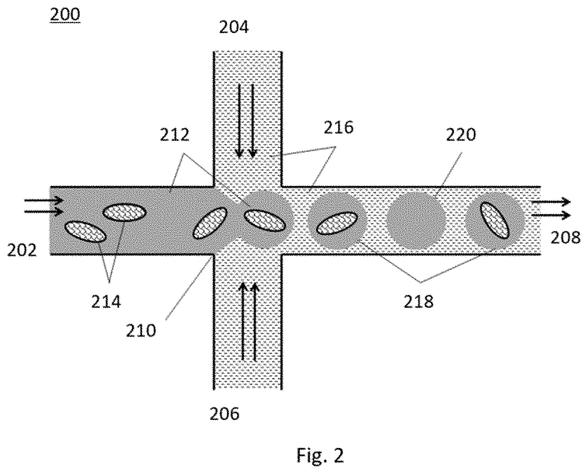

Briefly, the provision of discrete droplets in an emulsion encapsulating a biological sample (i.e., a biological particle) is accomplished by introducing a flowing stream of an aqueous fluid containing the biological sample (i.e., biological particles) into a flowing stream or reservoir of a non-aqueous fluid with which it is immiscible, such that droplets are generated (see generally, e.g., FIGS. 2-7). By providing the aqueous stream at a certain concentration and/or flow rate of the biological sample, the occupancy of the resulting droplets can be controlled. For example, the relative flow rates of the immiscible fluids can be selected such that, on average, the discrete droplet each contains less than one biological particle (e.g., one cell or one nucleus). Such a flow rate ensures that the droplets that are occupied are primarily occupied by a single biological particle (e.g., a single cell or a single nucleus). In some cases, the droplets among a plurality of discrete droplets formed in the manner contain at most one particle (e.g., one bead, one cell or one nucleus). The flows and microfluidic channel architectures also can be controlled to ensure a given number of singly occupied droplets, less than a certain level of unoccupied droplets, and/or less than a certain level of multiply occupied droplets.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

FIG. 2 shows an exemplary microfluidic channel structure 200 useful for generating discrete droplets encapsulating a particle from a biological sample, such as a single cell. The channel structure 200 can include channel segments 202, 204, 206 and 208 communicating at a channel junction 210. In operation, a first aqueous fluid 212 that that includes suspended particles (e.g., cells or nuclei) from a biological sample 214 are transported along channel segment 202 into junction 210, while a second fluid 216 (or “partitioning fluid”) that is immiscible with the aqueous fluid 212 is delivered to the junction 210 from each of channel segments 204 and 206 to create discrete droplets 218, 220 of the first 208, and flowing away from junction 210. The channel segment 208 may be fluidically coupled to an outlet reservoir where the discrete droplets can be stored and/or harvested. A discrete droplet generated may include an individual particle from a biological sample 214 (such as droplet 218), or discrete droplet can be generated that includes more than one particle 214 (not shown in FIG. 2). A discrete droplet may contain no biological particle 214 (such as droplet 220). Each discrete droplet is capable of maintaining separation of its own contents (e.g., individual biological sample particle 214) from the contents of other droplets.

Typically, the second fluid 216 comprises an oil, such as a fluorinated oil, that includes a fluoro-surfactant that helps to stabilize the resulting droplets. Examples of useful partitioning fluids and fluoro-surfactants are described in e.g., U.S. Patent Application Publication No. 2010/0105112, which is entirely incorporated herein by reference for all purposes.

The microfluidic channels for generating discrete droplets as exemplified in FIG. 2 may be coupled to any of a variety of different fluid sources or receiving components, including reservoirs, tubing, manifolds, or fluidic components of other systems. Additionally, the microfluidic channel structure 200 may have other geometries, including geometries having more than one channel junction. For example, the microfluidic channel structure can have 2, 3, 4, or 5 channel segments each carrying biological particles (e.g., cells or nuclei) from a sample, assay reagents, and/or gel beads that meet at a channel junction.

Generally, the fluids used in generating the discrete droplets are directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electro-kinetic pumping, vacuum, capillary or gravity flow, or the like.

One of ordinary skill will recognize that numerous different microfluidic channel designs are available that can be used with the methods and compositions of the present disclosure to provide discrete droplets containing a fixed biological particle (e.g., a cell or nucleus), and/or a bead with a barcode and/or other assay reagents.

The inclusion of a barcode in a discrete droplet along with the biological sample provides a unique identifier that allows data from the biological sample to be distinguished and individually analyzed. Barcodes can be delivered previous to, subsequent to, or concurrent with the biological sample in discrete droplet. For example, barcodes may be injected into droplets previous to, subsequent to, or concurrently with droplet generation. Barcodes useful in the methods and compositions of the present disclosure typically comprise a nucleic acid molecule (e.g., an oligonucleotide). The nucleic acid barcode molecules typically are delivered to a partition via a microcapsule, such as bead. In some cases, barcode nucleic acid molecules are initially associated with the bead upon generation of the discrete droplet, and then released from the bead upon application of a stimulus to droplet. Barcode carrying beads useful in the methods and compositions of the present disclosure are described in further detail elsewhere herein.

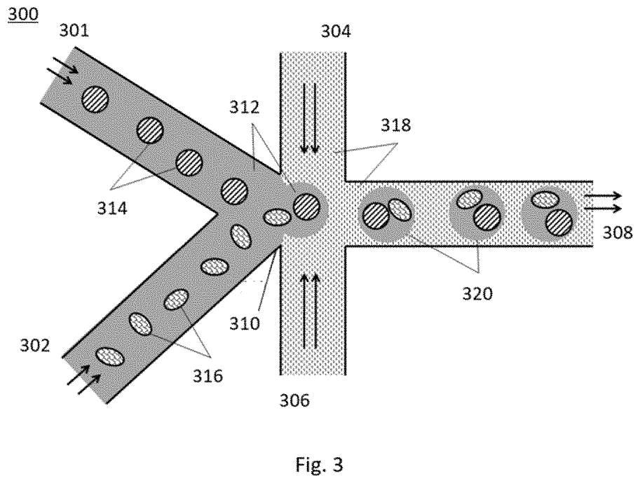

FIG. 3 shows an exemplary microfluidic channel structure 300 for generating discrete droplets encapsulating a barcode carrying bead 314 along with a biological sample particle 316. The channel structure 230 includes channel segments 301, 302, 304, 306 and 308 in fluid communication at a channel junction 310. In operation, the channel segment 301 transports an aqueous fluid 312 that can include a plurality of beads 314 (e.g., gel beads carrying barcode oligonucleotides) along the channel segment 301 into junction 310. The plurality of beads 314 may be sourced from a suspension of beads. For example, the channel segment 301 can be connected to a reservoir comprising an aqueous suspension of beads 314. The channel segment 302 transports the aqueous fluid 312 that includes a plurality of biological sample particles 316 along the channel segment 302 into junction 310. The plurality of biological sample particles 316 may be sourced from a suspension of biological sample particles. For example, the channel segment 302 may be connected to a reservoir comprising an aqueous suspension of biological sample particles 316. In some instances, the aqueous fluid 312 in either the first channel segment 301 or the second channel segment 302, or in both segments, can include one or more reagents, as further described elsewhere herein. For example, in some embodiments of the present disclosure, where the biological sample particles are fixed biological sample particles, the aqueous fluid in the first and/or second channel segments that delivers the biological sample and beads, respectively. The second fluid 318 that is immiscible with the aqueous fluid 312 is delivered to the junction 310 from each of channel segments 304 and 306. Upon meeting of the aqueous fluid 312 from each of channel segments 301 and 302 and the second fluid 318 (e.g., a fluorinated oil) from each of channel segments 304 and 306 at the channel junction 310, the aqueous fluid 312 is partitioned into discrete droplets 320 in the second fluid 318 and flow away from the junction 310 along channel segment 308. The channel segment 308 can then deliver the discrete droplets encapsulating the biological sample particle and barcode carrying bead to an outlet reservoir fluidly coupled to the channel segment 308, where they can be collected.

As an alternative, the channel segments 301 and 302 may meet at another junction upstream of the junction 310. At such junction, beads and biological particles may form a mixture that is directed along another channel to the junction 310 to yield droplets 320. The mixture may provide the beads and biological particles in an alternating fashion, such that, for example, a droplet comprises a single bead and a single biological particle.

Using such a channel system as exemplified in FIG. 3, discrete droplets 320 can be generated that encapsulate an individual particle of a biological sample, and one bead, wherein the bead can carry a barcode and/or another reagent. It is also contemplated, that in some instances, a discrete droplet may be generated using the channel system of FIG. 3, wherein droplet includes more than one individual biological sample particle or includes no biological sample. Similarly, in some embodiments, the discrete droplet may include more than one bead or no bead. A discrete droplet also may be completely unoccupied (e.g., no bead or biological sample).

In some embodiments, it is desired that the beads, single cells or nuclei, and generated discrete droplets flow along channels at substantially regular flow rates that generate a discrete droplet containing a single bead and a single biological sample particle. Regular flow rates and devices that may be used to provide such regular flow rates are known in the art, see e.g., U.S. Patent Publication No. 2015/0292988, which is hereby incorporated by reference herein in its entirety. In some embodiments, the flow rates are set to provide discrete droplets containing a single bead and a biological sample particle with a yield rate of greater than 5%, 10%, 20%, 30%, 40%, 50%, 60%, 70%, 80%, 90%, or 95%.

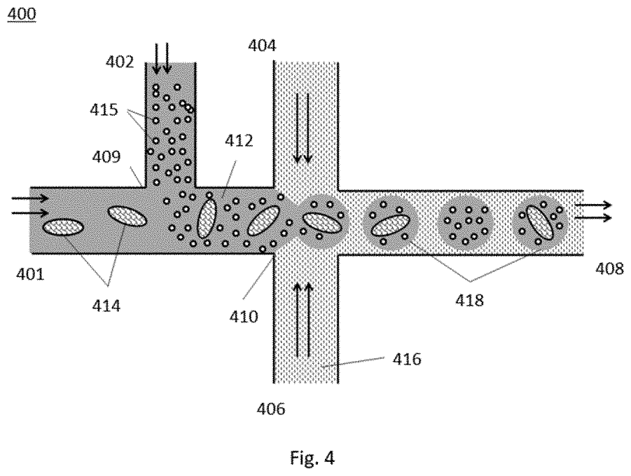

In some embodiments, a biological sample particle can be co-partitioned along with the other reagents. FIG. 4 shows an example of a microfluidic channel structure 400 for co-partitioning biological sample particles and other reagents, including lysis agents. The channel structure 400 can include channel segments 401, 402, 404, 406 and 408. Channel segments 401 and 402 communicate at a first channel junction 409. Channel segments 402, 404, 406, and 408 communicate at a second channel junction 410. In exemplary co-partitioning operation, the channel segment 401 may transport an aqueous fluid 412 that includes a plurality of biological sample particles 414 (e.g., a fixed biological sample) along the channel segment 401 into the second junction 410. As an alternative or in addition to, channel segment 401 may transport beads (e.g., gel beads that carry barcodes). For example, the channel segment 401 may be connected to a reservoir comprising an aqueous suspension of biological sample particles 414. Upstream of, and immediately prior to reaching, the second junction 410, the channel segment 401 may meet the channel segment 402 at the first junction 409. The channel segment 402 can transport a plurality of reagents 415 (e.g., lysis agents) in the aqueous fluid 412 along the channel segment 402 into the first junction 409. For example, the channel segment 402 may be connected to a reservoir comprising the reagents 415. After the first junction 409, the aqueous fluid 412 in the channel segment 401 can carry both the biological sample particles 414 and the reagents 415 towards the second junction 410. In some instances, the aqueous fluid 412 in the channel segment 401 can include one or more reagents, which can be the same or different reagents as the reagents 415. A second fluid 416 that is immiscible with the aqueous fluid 412 (e.g., a fluorinated oil) can be delivered to the second junction 410 from each of channel segments 404 and 406. Upon meeting of the aqueous fluid 412 from the channel segment 401 and the second fluid 416 from each of channel segments 404 and 406 at the second channel junction 410, the aqueous fluid 412 is partitioned as discrete droplets 418 in the second fluid 416 and flow away from the second junction 410 along channel segment 408. The channel segment 408 may deliver the discrete droplets 418 to an outlet reservoir fluidly coupled to the channel segment 408, where they may be collected for further analysis.

Discrete droplets generated can include an individual biological sample particle 414 and/or one or more reagents 415, depending on what reagents are included in channel segment 402. In some instances, a discrete droplet generated may also include a barcode carrying bead (not shown), such as can be added via other channel structures described elsewhere herein. In some instances, a discrete droplet may be unoccupied (e.g., no reagents, no biological particles). Generally, the channel segments described herein may be coupled to any of a variety of different fluid sources or receiving components, including reservoirs, tubing, manifolds, or fluidic components of other systems. As will be appreciated, the microfluidic channel structure 400 may have other geometries. For example, a microfluidic channel structure can have more than two channel junctions. For example, a microfluidic channel structure can have 2, 3, 4, 5 channel segments or more each carrying the same or different types of beads, reagents, and/or biological sample particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electro-kinetic pumping, vacuum, capillary or gravity flow, or the like.

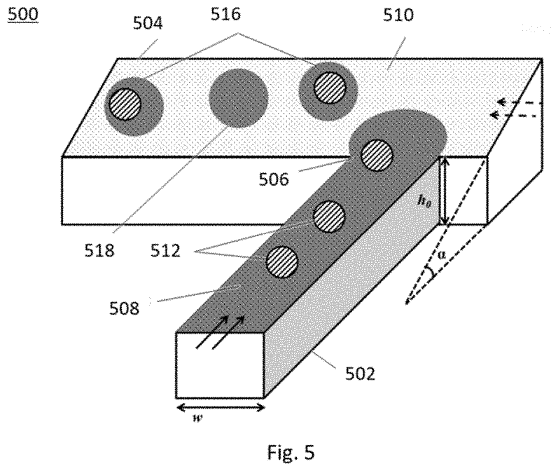

FIG. 5 shows an example of a microfluidic channel structure for the controlled partitioning of beads into discrete droplets. A channel structure 500 can include a channel segment 502 communicating at a channel junction 506 (or intersection) with a reservoir 504. The reservoir 504 can be a chamber. Any reference to “reservoir,” as used herein, can also refer to a “chamber.” In operation, an aqueous fluid 508 that includes suspended beads 512 may be transported along the channel segment 502 into the junction 506 to meet a second fluid 510 that is immiscible with the aqueous fluid 508 in the reservoir 504 to create droplets 516, 518 of the aqueous fluid 508 flowing into the reservoir 504. At the junction 506 where the aqueous fluid 508 and the second fluid 510 meet, droplets can form based on factors such as the hydrodynamic forces at the junction 506, flow rates of the two fluids 508, 510, fluid properties, and certain geometric parameters (e.g., w, h0, α, etc.) of the channel structure 500. A plurality of droplets can be collected in the reservoir 504 by continuously injecting the aqueous fluid 508 from the channel segment 502 through the junction 506.

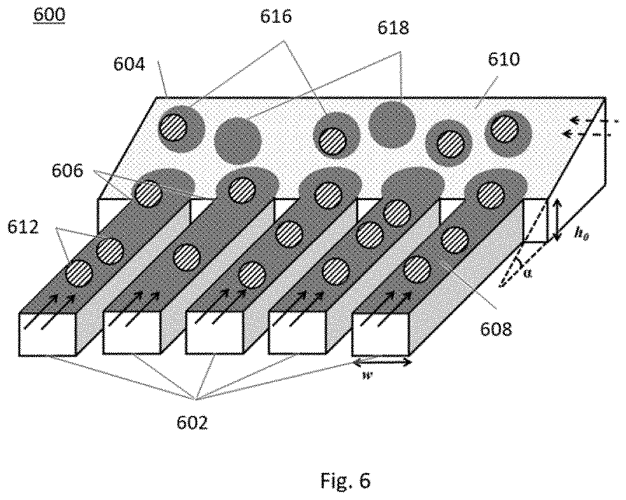

FIG. 6 shows an example of a microfluidic channel structure for increased droplet generation throughput. A microfluidic channel structure 600 can comprise a plurality of channel segments 602 and a reservoir 604. Each of the plurality of channel segments 602 may be in fluid communication with the reservoir 604. The channel structure 600 can comprise a plurality of channel junctions 606 between the plurality of channel segments 602 and the reservoir 604. Each channel junction can be a point of droplet generation. The channel segment 602 from the channel structure 500 in FIG. 5 and any description to the components thereof may correspond to a given channel segment of the plurality of channel segments 602 in channel structure 600 and any description to the corresponding components thereof. The reservoir 504 from the channel structure 500 and any description to the components thereof may correspond to the reservoir 604 from the channel structure 600 and any description to the corresponding components thereof.

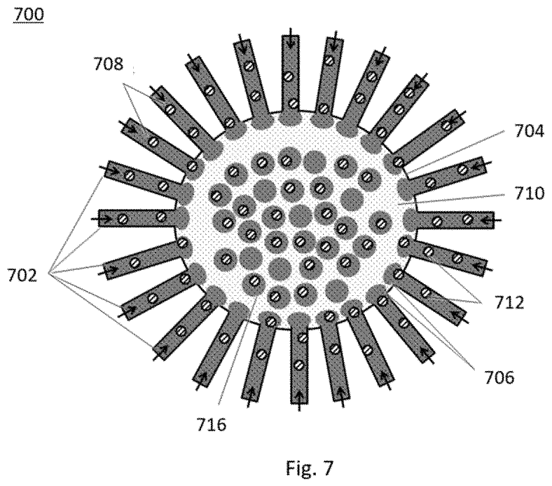

FIG. 7 shows another example of a microfluidic channel structure for increased droplet generation throughput. A microfluidic channel structure 700 can comprise a plurality of channel segments 702 arranged generally circularly around the perimeter of a reservoir 704. Each of the plurality of channel segments 702 may be in fluid communication with the reservoir 704. The channel structure 700 can comprise a plurality of channel junctions 706 between the plurality of channel segments 702 and the reservoir 704. Each channel junction can be a point of droplet generation. The channel segment 502 from the channel structure 500 in FIG. 5 and any description to the components thereof may correspond to a given channel segment of the plurality of channel segments 702 in channel structure 700 and any description to the corresponding components thereof. The reservoir 504 from the channel structure 500 and any description to the components thereof may correspond to the reservoir 704 from the channel structure 700 and any description to the corresponding components thereof. Additional aspects of such the microfluidic structures depicted in FIGS. 5-7, including systems and methods implementing the same, are provided in US Published Patent Application No 20190323088, which is incorporated herein by reference in its entirety.

### Microwell-Based Analysis

As described herein, one or more processes can be performed in a partition, which can be a well. The well can be a well of a plurality of wells of a substrate, such as a microwell of a microwell array or plate, or the well can be a microwell or microchamber of a device (e.g., microfluidic device) comprising a substrate. The well can be a well of a well array or plate, or the well can be a well or chamber of a device (e.g., fluidic device). Accordingly, the wells or microwells can assume an “open” configuration, in which the wells or microwells are exposed to the environment (e.g., contain an open surface) and are accessible on one planar face of the substrate, or the wells or microwells can assume a “closed” or “sealed” configuration, in which the microwells are not accessible on a planar face of the substrate. In some instances, the wells or microwells can be configured to toggle between “open” and “closed” configurations. For instance, an “open” microwell or set of microwells can be “closed” or “sealed” using a membrane (e.g., semi-permeable membrane), an oil (e.g., fluorinated oil to cover an aqueous solution), or a lid, as described elsewhere herein. The wells or microwells can be initially provided in a “closed” or “sealed” configuration, wherein they are not accessible on a planar surface of the substrate without an external force. For instance, the “closed” or “sealed” configuration can include a substrate such as a sealing film or foil that is puncturable or pierceable by pipette tip(s). Suitable materials for the substrate include, without limitation, polyester, polypropylene, polyethylene, vinyl, and aluminum foil.

In some embodiments, the well can have a volume of less than 1 milliliter (mL). For example, the well can be configured to hold a volume of at most 1000 microliters (μL), at most 100 μL, at most 10 μL, at most 1 μL, at most 100 nanoliters (nL), at most 10 nL, at most 1 nL, at most 100 picoliters (μL), at most 10 (μL), or less. The well can be configured to hold a volume of about 1000 μL, about 100 μL, about 10 μL, about 1 μL, about 100 nL, about 10 nL, about 1 nL, about 100 μL, about 10 μL, etc. The well can be configured to hold a volume of at least 10 μL, at least 100 μL, at least 1 nL, at least 10 nL, at least 100 nL, at least 1 μL, at least 10 μL, at least 100 μL, at least 1000 μL, or more. The well can be configured to hold a volume in a range of volumes listed herein, for example, from about 5 nL to about 20 nL, from about 1 nL to about 100 nL, from about 500 μL to about 100 μL, etc. The well can be of a plurality of wells that have varying volumes and can be configured to hold a volume appropriate to accommodate any of the partition volumes described herein.

In some instances, a microwell array or plate includes a single variety of microwells. In some instances, a microwell array or plate includes a variety of microwells. For instance, the microwell array or plate can include one or more types of microwells within a single microwell array or plate. The types of microwells can have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate can include any number of different types of microwells. For example, the microwell array or plate can include 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well can have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate includes different types of microwells that are located adjacent to one another within the array or plate. For example, a microwell with one set of dimensions can be located adjacent to and in contact with another microwell with a different set of dimensions. Similarly, microwells of different geometries can be placed adjacent to or in contact with one another. The adjacent microwells can be configured to hold different articles; for example, one microwell can be used to contain a biological particle, such as a cell, a nucleus, or other sample (e.g., cellular components, nucleic acid molecules, etc.) while the adjacent microwell can be used to contain a support (e.g., a bead such as a gel bead), droplet, or other reagent. In some cases, the adjacent microwells can be configured to merge the contents held within, e.g., upon application of a stimulus, or spontaneously, upon contact of the articles in each microwell.

As is described elsewhere herein, a plurality of partitions can be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells can include both unoccupied wells (e.g., empty wells) and occupied wells.

A well can include any of the reagents described herein, or combinations thereof. These reagents can include, for example, barcode molecules, enzymes, adapters, and combinations thereof. The reagents can be physically separated from a biological particle (for example, a cell, a nucleus, or cellular components, e.g., proteins, nucleic acid molecules, etc.) that is placed in the well. This physical separation can be accomplished by containing the reagents within, or coupling to, a support (e.g., a bead such as a gel bead) that is placed within a well. The physical separation can also be accomplished by dispensing the reagents in the well and overlaying the reagents with a layer that is, for example, dissolvable, meltable, or permeable prior to introducing the polynucleotide sample into the well. This layer can be, for example, an oil, wax, membrane (e.g., semi-permeable membrane), or the like. The well can be sealed at any point, for example, after addition of the support or bead, after addition of the reagents, or after addition of either of these components. The sealing of the well can be useful for a variety of purposes, including preventing escape of beads or loaded reagents from the well, permitting select delivery of certain reagents (e.g., via the use of a semi-permeable membrane), for storage of the well prior to or following further processing, etc.

A well can include free reagents and/or reagents encapsulated in, or otherwise coupled to or associated with, supports (e.g., beads), or droplets. In some embodiments, any of the reagents described in this disclosure can be encapsulated in, or otherwise coupled to, a support (e.g., a bead) or a droplet, with any chemicals, particles, and elements suitable for sample processing reactions involving biomolecules, such as, but not limited to, nucleic acid molecules and proteins. For example, a bead or droplet used in a sample preparation reaction for DNA sequencing can include one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, oligonucleotides, nucleotides, deoxyribonucleotide triphosphates (dNTPs), dideoxyribonucleotide triphosphates (ddNTPs), DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA, polymerase, ligase, restriction enzymes, proteases, nucleases, protease inhibitors, nuclease inhibitors, chelating agents, reducing agents, oxidizing agents, fluorophores, probes, chromophores, dyes, organics, emulsifiers, surfactants, stabilizers, polymers, water, small molecules, pharmaceuticals, radioactive molecules, preservatives, antibiotics, aptamers, and pharmaceutical drug compounds. As described herein, one or more reagents in the well can be used to perform one or more reactions, including but not limited to: biological particle (e.g., a cell or a nucleus) processing such as lysis, fixation, permeabilization, nucleic acid reactions, e.g., nucleic acid extension reactions, amplification, reverse transcription, reactions, etc.

The wells disclosed herein can be provided as a part of a kit. For example, a kit can include instructions for use, a microwell array or device, and reagents (e.g., beads). The kit can include any useful reagents for performing the processes described herein, e.g., nucleic acid reactions, barcoding of nucleic acid molecules, sample processing (e.g., for biological particle lysis, fixation, and/or permeabilization).

In some cases, a well includes a support (e.g., a bead) or droplet that includes a set of reagents that has a similar attribute, for example, a set of enzymes, a set of minerals, a set of oligonucleotides, a mixture of different barcode molecules, or a mixture of identical barcode molecules. In other cases, a support (e.g., a bead) or droplet includes a heterogeneous mixture of reagents. In some cases, the heterogeneous mixture of reagents can include all components necessary to perform a reaction. In some cases, such mixture can include all components necessary to perform a reaction, except for 1, 2, 3, 4, 5, or more components necessary to perform a reaction. In some cases, such additional components are contained within, or otherwise coupled to, a different support (e.g., a bead) or droplet, or within a solution within a partition (e.g., microwell) of the system.

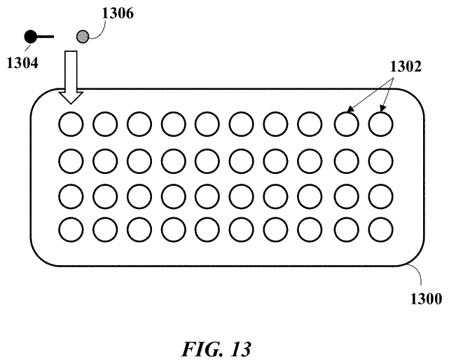

A non-limiting example of a microwell array in accordance with some embodiments of the disclosure is schematically presented in FIG. 13. In this example, the array can be contained within a substrate 1300. The substrate 1300 includes a plurality of wells 1302. The wells 1302 can be of any size or shape, and the spacing between the wells, the number of wells per substrate, as well as the density of the wells on the substrate 1300 can be modified, depending on the particular application. In one such example application, a sample molecule 1306, which can include a biological particle, such as a cell, a nucleus, or cellular components (e.g., nucleic acid molecules) is co-partitioned with a bead 1304, which can include a nucleic acid barcode molecule coupled thereto. The wells 1302 can be loaded using gravity or other loading technique (e.g., centrifugation, liquid handler, acoustic loading, optoelectronic, etc.). In some instances, at least one of the wells 1302 contains a single biological particle 1306 (e.g., cell or a nucleus) and a single bead 1304.

Reagents can be loaded into a well either sequentially or concurrently. In some cases, reagents are introduced to the device either before or after a particular operation. In some cases, reagents (which can be provided, in certain instances, in supports (e.g., beads) or droplets) are introduced sequentially such that different reactions or operations occur at different steps. The reagents (or supports (e.g., beads) or droplets) can also be loaded at operations interspersed with a reaction or operation step. For example, supports (e.g., beads) (or droplets) including reagents for fragmenting polynucleotides (e.g., restriction enzymes) and/or other enzymes (e.g., ligases, polymerases, etc.) can be loaded into the well or plurality of wells, followed by loading of supports (e.g., beads) or droplets, including reagents for attaching nucleic acid barcode molecules to a sample nucleic acid molecule. Reagents can be provided concurrently or sequentially with a biological particle, e.g., a cell, a nucleus, or cellular components (e.g., organelles, proteins, nucleic acid molecules, carbohydrates, lipids, etc.). Accordingly, use of wells can be useful in performing multi-step operations or reactions.

As described elsewhere herein, the nucleic acid barcode molecules and other reagents can be contained within a support (e.g., a bead such as a gel bead) or droplet. These supports or droplets can be loaded into a partition (e.g., a microwell) before, after, or concurrently with the loading of a biological particle (e.g. a cell or a nucleus), such that each biological particle is contacted with a different support (e.g., bead), or droplet. This technique can be used to attach a unique nucleic acid barcode molecule to nucleic acid molecules obtained from each biological particle. Alternatively or in addition, the sample nucleic acid molecules can be attached to a support. For example, the partition (e.g., microwell) can include a bead which has coupled thereto a plurality of nucleic acid barcode molecules. The sample nucleic acid molecules, or derivatives thereof, can couple or attach to the nucleic acid barcode molecules attached on the support. The resulting barcoded nucleic acid molecules can then be removed from the partition, and in some instances, pooled and sequenced. In such cases, the nucleic acid barcode sequences can be used to trace the origin of the sample nucleic acid molecule. For example, polynucleotides with identical barcodes can be determined to originate from the same biological particle or partition, while polynucleotides with different barcodes can be determined to originate from different biological particles (e.g., cells or nuclei) or partitions.

The samples or reagents can be loaded in the wells or microwells using a variety of approaches. For example, the biological particles (e.g., a cell, a nucleus, or cellular component) or reagents (as described herein) can be loaded into the well or microwell using an external force, e.g., gravitational force, electrical force, magnetic force, or using mechanisms to drive the sample or reagents into the well, for example, via pressure-driven flow, centrifugation, optoelectronics, acoustic loading, electrokinetic pumping, vacuum, capillary flow, etc. In certain cases, a fluid handling system can be used to load the samples or reagents into the well. The loading of the samples or reagents can follow a Poissonian distribution or a non-Poissonian distribution, e.g., super Poisson or sub-Poisson. The geometry, spacing between wells, density, and size of the microwells can be modified to accommodate a useful sample or reagent distribution; for example, the size and spacing of the microwells can be adjusted such that the sample or reagents can be distributed in a super-Poissonian fashion.

In one non-limiting example, the microwell array or plate includes pairs of microwells, in which each pair of microwells is configured to hold a droplet (e.g., including a single biological particle, such as a cell or a nucleus) and a single bead (such as those described herein, which can, in some instances, also be encapsulated in a droplet). The droplet and the bead (or droplet containing the bead) can be loaded simultaneously or sequentially, and the droplet and the bead can be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets including different reagents and/or samples, which are merged upon contact or upon application of a stimulus. In such instances, the droplet of one microwell of the pair can include reagents that can react with an agent in the droplet of the other microwell of the pair. For example, one droplet can include reagents that are configured to release the nucleic acid barcode molecules of a bead contained in another droplet, located in the adjacent microwell. Upon merging of the droplets, the nucleic acid barcode molecules can be released from the bead into the partition (e.g., the microwell or microwell pair that are in contact), and further processing can be performed (e.g., barcoding, nucleic acid reactions, etc.). In cases where intact or live biological particles (e.g., cells) are loaded in the microwells, one of the droplets can include lysis reagents for lysing the biological particle (e.g., cell) upon droplet merging.

In some embodiments, a droplet or support can be partitioned into a well. The droplets can be selected or subjected to pre-processing prior to loading into a well. For instance, the droplets can include biological particles (e.g., cells or nuclei), and only certain droplets, such as those containing a single biological particle (e.g., a cell or a nucleus) (or at least one biological particle such as a cell or nucleus), can be selected for use in loading of the wells. Such a pre-selection process can be useful in efficient loading of single biological particles, such as to obtain a non-Poissonian distribution, or to pre-filter biological particles (e.g., cells or nuclei) for a selected characteristic prior to further partitioning in the wells. Additionally, the technique can be useful in obtaining or preventing biological particle (e.g., a cell or nucleus) doublet or multiplet formation prior to or during loading of the microwell.

In some embodiments, the wells can include nucleic acid barcode molecules attached thereto. The nucleic acid barcode molecules can be attached to a surface of the well (e.g., a wall of the well). The nucleic acid barcode molecule (e.g., a partition barcode sequence) of one well can differ from the nucleic acid barcode molecule of another well, which can permit identification of the contents contained with a single partition or well. In some embodiments, the nucleic acid barcode molecule can include a spatial barcode sequence that can identify a spatial coordinate of a well, such as within the well array or well plate. In some embodiments, the nucleic acid barcode molecule can include a unique molecular identifier for individual molecule identification. In some instances, the nucleic acid barcode molecules can be configured to attach to or capture a nucleic acid molecule within a sample or a biological particle (e.g., a cell or nucleus) distributed in the well. For example, the nucleic acid barcode molecules can include a capture sequence that can be used to capture or hybridize to a nucleic acid molecule (e.g., RNA, DNA) within the sample or a nucleic acid complex (e.g., a complex comprising probes hybridized to a sample nucleic acid molecule). In some embodiments, the nucleic acid barcode molecules can be releasable from the microwell. For example, the nucleic acid barcode molecules can include a chemical cross-linker which can be cleaved upon application of a stimulus (e.g., photo-, magnetic, chemical, biological, stimulus). The released nucleic acid barcode molecules, which can be hybridized or configured to hybridize to a sample nucleic acid molecule, can be collected and pooled for further processing, which can include nucleic acid processing (e.g., amplification, extension, reverse transcription, etc.) and/or characterization (e.g., sequencing). In such cases, the unique partition barcode sequences can be used to identify the biological particle or partition from which a nucleic acid molecule originated.

Characterization of samples within a well can be performed. Such characterization can include, in non-limiting examples, imaging of the biological particle (e.g., cell, nucleus, or cellular components) or derivatives thereof. Characterization techniques such as microscopy or imaging can be useful in measuring sample profiles in fixed spatial locations. For example, when biological particles are partitioned, optionally with beads, imaging of each microwell and the contents contained therein can provide useful information on biological particle (e.g., cell or nucleus) doublet formation (e.g., frequency, spatial locations, etc.), viability, size, morphology, expression level of a biomarker (e.g., a surface marker, a fluorescently labeled molecule therein, etc.), biological particle or bead loading rate, number of biological particle-bead pairs, etc. In some instances, imaging can be used to characterize live cells in the wells, including, but not limited to: dynamic live-cell tracking, cell-cell interactions (when two or more cells are co-partitioned), cell proliferation, etc. Alternatively or in addition to, imaging can be used to characterize a quantity of amplification products in the well.

In operation, a well can be loaded with a sample and reagents, simultaneously or sequentially. When biological particles (e.g., cells or nuclei) are loaded, the well can be subjected to washing, e.g., to remove excess biological particles from the well, microwell array, or plate. Similarly, washing can be performed to remove excess beads or other reagents from the well, microwell array, or plate. In the instances where live cells are used, the cells can be lysed in the individual partitions to release the intracellular components or cellular analytes. Alternatively, the cells can be fixed or permeabilized in the individual partitions. The intracellular components or cellular analytes can couple to a support, e.g., on a surface of the microwell, on a solid support (e.g., bead), or they can be collected for further downstream processing. For example, after cell lysis, the intracellular components or cellular analytes can be transferred to individual droplets or other partitions for barcoding. Alternatively, or in addition, the intracellular components or cellular analytes (e.g., nucleic acid molecules) can couple to a bead including a nucleic acid barcode molecule; subsequently, the bead can be collected and further processed, e.g., subjected to nucleic acid reaction such as reverse transcription, amplification, or extension, and the nucleic acid molecules thereon can be further characterized, e.g., via sequencing. Alternatively, or in addition, the intracellular components or cellular analytes can be barcoded in the well (e.g., using a bead including nucleic acid barcode molecules that are releasable or on a surface of the microwell including nucleic acid barcode molecules). The barcoded nucleic acid molecules or analytes can be further processed in the well, or the barcoded nucleic acid molecules or analytes can be collected from the individual partitions and subjected to further processing outside the partition. Further processing can include nucleic acid processing (e.g., performing an amplification, extension) or characterization (e.g., fluorescence monitoring of amplified molecules, sequencing). At any suitable or useful step, the well (or microwell array or plate) can be sealed (e.g., using an oil, membrane, wax, etc.), which enables storage of the assay or selective introduction of additional reagents.

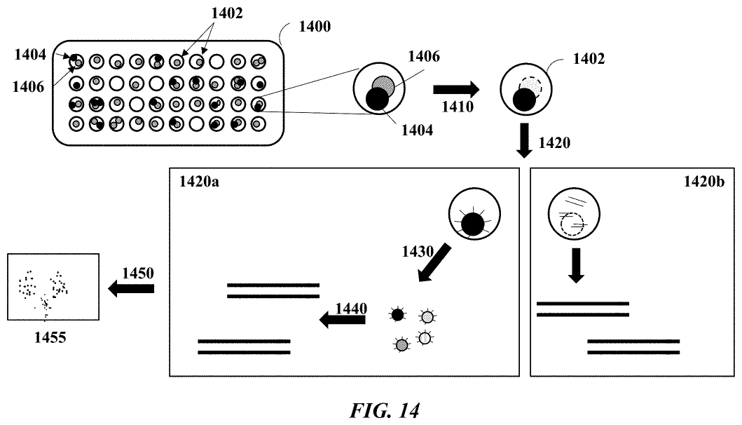

FIG. 14 schematically shows an example workflow for processing nucleic acid molecules within a sample. A substrate 1400 including a plurality of microwells 1402 can be provided. A sample 1406 which can include a biological particle (e.g., a cell, a nucleus, cellular components or analytes (e.g., proteins and/or nucleic acid molecules) can be co-partitioned, in a plurality of microwells 1402, with a plurality of beads 1404 including nucleic acid barcode molecules. During a partitioning process, the sample 1406 can be processed within the partition. For instance, in the case of live cells, the cell can be subjected to conditions sufficient to lyse the cells and release the analytes contained therein. In process 1420, the bead 1404 can be further processed. By way of example, processes 1420a and 1420b schematically illustrate different workflows, depending on the properties of the bead 1404.

In 1420a, the bead includes nucleic acid barcode molecules that are attached thereto, and sample nucleic acid molecules (e.g., RNA, DNA) can attach, e.g., via hybridization of ligation, to the nucleic acid barcode molecules. Such attachment can occur on the bead. In process 1430, the beads 1404 from multiple wells 1402 can be collected and pooled. Further processing can be performed in process 1440. For example, one or more nucleic acid reactions can be performed, such as reverse transcription, nucleic acid extension, amplification, ligation, etc. In some instances, adapter sequences are ligated to the nucleic acid molecules, or derivatives thereof, as described elsewhere herein. For instance, sequencing primer sequences can be appended to each end of the nucleic acid molecule. In process 1450, further characterization, such as sequencing can be performed to generate sequencing reads. The sequencing reads can yield information on individual biological particles (e.g., cells or nuclei) or populations of biological particles, which can be represented visually or graphically, e.g., in a plot.

In 1420b, the bead includes nucleic acid barcode molecules that are releasably attached thereto, as described below. The bead can degrade or otherwise release the nucleic acid barcode molecules into the well 1402; the nucleic acid barcode molecules can then be used to barcode nucleic acid molecules within the well 1402. Further processing can be performed either inside the partition or outside the partition. For example, one or more nucleic acid reactions can be performed, such as reverse transcription, nucleic acid extension, amplification, ligation, etc. In some instances, adapter sequences are ligated to the nucleic acid molecules, or derivatives thereof, as described elsewhere herein. For instance, sequencing primer sequences can be appended to each end of the nucleic acid molecule. In process 1450, further characterization, such as sequencing can be performed to generate sequencing reads. The sequencing reads can yield information on individual cells or populations of cells, which can be represented visually or graphically, e.g., in a plot.

### Multiplexing Methods

In some embodiments of the disclosure, the methods described herein can be performed in multiplex format. Accordingly, in some embodiments, the present disclosure provides methods and systems for multiplexing, and otherwise increasing throughput of samples for analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more biological particles or features of biological particles (e.g., cells or cell features, as well as nuclei or nuclei features) can be used to characterize the same. In some instances, cell or nucleus features include cell surface or nucleus surface (e.g., nuclear membrane) features. Cell or nuclear surface features can include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell or nucleus features can include intracellular analytes, such as proteins, protein modifications (e.g., phosphorylation status or other post-translational modifications), nuclear proteins, nuclear membrane proteins, or any combination thereof. A labelling agent can include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a Darpin, and a protein scaffold, or any combination thereof. The labelling agents can include (e.g., are attached to) a reporter oligonucleotide that is indicative of the cell surface feature to which the binding group binds. For example, the reporter oligonucleotide can include a barcode sequence that permits identification of the labelling agent. For example, a labelling agent that is specific to one type of cell or nucleus feature (e.g., a first cell or nucleus surface feature) can have a first reporter oligonucleotide coupled thereto, while a labelling agent that is specific to a different cell or nucleus feature (e.g., a second cell or nucleus surface feature) can have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969.

In a particular example, a library of potential cell or nucleus feature labelling agents can be provided, where the respective cell or nucleus feature labelling agents are associated with nucleic acid reporter molecules, such that a different reporter oligonucleotide sequence is associated with each labelling agent capable of binding to a specific cell or nucleus feature. In other aspects, different members of the library can be characterized by the presence of a different oligonucleotide sequence label. For example, an antibody capable of binding to a first protein can have associated with it a first reporter oligonucleotide sequence, while an antibody capable of binding to a second protein can have a different reporter oligonucleotide sequence associated with it. The presence of the particular oligonucleotide sequence can be indicative of the presence of a particular antibody or cell/nucleus feature which can be recognized or bound by the particular antibody.

Labelling agents capable of binding to or otherwise coupling to one or more cells or nuclei can be used to characterize a cell or nucleus as belonging to a particular set of cells. For example, labeling agents can be used to label a sample of cells/nuclei or a group of cells or nuclei. In this way, a group of cells/nuclei can be labeled as different from another group of cells/nuclei. In an example, a first group of cells/nuclei can originate from a first sample and a second group of cells/nuclei can originate from a second sample. Labelling agents can allow the first group and second group to have a different labeling agent (or reporter oligonucleotide associated with the labeling agent). This can, for example, facilitate multiplexing, where cells/nuclei of the first group and cells/nuclei of the second group can be labeled separately and then pooled together for downstream analysis. The downstream detection of a label can indicate analytes as belonging to a particular group.

For example, a reporter oligonucleotide can be linked to an antibody or an epitope binding fragment thereof, and labeling a cell or nucleus can include subjecting the antibody-linked barcode molecule or the epitope binding fragment-linked barcode molecule to conditions suitable for binding the antibody to a molecule present on a surface of the cell or nucleus. The binding affinity between the antibody or the epitope binding fragment thereof and the molecule present on the surface can be within a desired range to ensure that the antibody or the epitope binding fragment thereof remains bound to the molecule. For example, the binding affinity can be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds can be less than about 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, or 1 pM. For example, the dissociation constant can be less than about 10 μM.

In another example, a reporter oligonucleotide can be coupled to a cell-penetrating peptide (CPP), and labeling cells can include delivering the CPP coupled reporter oligonucleotide into an analyte carrier. Labeling analyte carriers can include delivering the CPP conjugated oligonucleotide into a cell or nucleus by the cell-penetrating peptide. A CPP that can be used in the methods provided herein can include at least one non-functional cysteine residue, which can be either free or derivatized to form a disulfide link with an oligonucleotide that has been modified for such linkage. Non-limiting examples of CPPs that can be used in embodiments herein include penetratin, transportan, plsl, TAT(48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The CPP can be an arginine-rich peptide transporter. The CPP can be Penetratin or the Tat peptide. In another example, a reporter oligonucleotide can be coupled to a fluorophore or dye, and labeling cells can include subjecting the fluorophore-linked barcode molecule to conditions suitable for binding the fluorophore to the surface of the cell. In some instances, fluorophores can interact strongly with lipid bilayers and labeling cells/nuclei can include subjecting the fluorophore-linked barcode molecule to conditions such that the fluorophore binds to or is inserted into a membrane of the cell or nucleus. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649 for a description of organic fluorophores.

A reporter oligonucleotide can be coupled to a lipophilic molecule, and labeling cells/nuclei can include delivering the nucleic acid barcode molecule to a membrane of a cell or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and the cell or nuclear membrane can be such that the membrane retains the lipophilic molecule (e.g., and associated components, such as nucleic acid barcode molecules, thereof) during subsequent processing (e.g., partitioning, cell permeabilization, amplification, pooling, etc.). The reporter nucleotide can enter into the intracellular space and/or a cell nucleus. In some embodiments, a reporter oligonucleotide coupled to a lipophilic molecule will remain associated with and/or inserted into lipid membrane (as described herein) via the lipophilic molecule until lysis of the cell or nucleus occurs, e.g., inside a partition. Exemplary embodiments of lipophilic molecules coupled to reporter oligonucleotides are described in PCT/US2018/064600.

A reporter oligonucleotide can be part of a nucleic acid molecule including any number of functional sequences, as described elsewhere herein, such as a target capture sequence, a random primer sequence, and the like, and coupled to another nucleic acid molecule that is, or is derived from, the analyte.

Prior to partitioning, the cells or nuclei can be incubated with the library of labelling agents, that can be labelling agents to a broad panel of different cell or nucleus features, e.g., receptors, proteins, etc., and which include their associated reporter oligonucleotides. Unbound labelling agents can be washed from the cells/nuclei, and the cells/nuclei can then be co-partitioned (e.g., into droplets or wells) along with partition-specific barcode oligonucleotides (e.g., attached to a support, such as a bead or gel bead) as described elsewhere herein. As a result, the partitions can include the cell/nucleus or cells/nuclei, as well as the bound labelling agents and their known, associated reporter oligonucleotides.

In other instances, e.g., to facilitate sample multiplexing, a labelling agent that is specific to a particular cell or nucleus feature can have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent can interact with different cells/nuclei, cell/nucleus populations or samples, allowing a particular report oligonucleotide to indicate a particular cell or nucleus population (or cell/nuclei or sample) and cell feature. In this way, different samples or groups can be independently processed and subsequently combined together for pooled analysis (e.g., partition-based barcoding as described elsewhere herein). See, e.g., U.S. Pat. Pub. 20190323088.

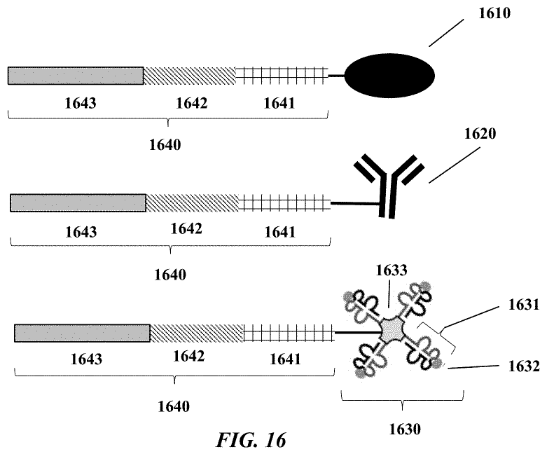

In some embodiments, to facilitate sample multiplexing, individual samples can be stained with lipid tags, such as cholesterol-modified oligonucleotides (CMOs, see, e.g., FIG. 16), anti-calcium channel antibodies, or anti-ACTB antibodies.

Non-limiting examples of anti-calcium channel antibodies include anti-KCNN4 antibodies, anti-BK channel beta 3 antibodies, anti-a1B calcium channel antibodies, and anti-CACNA1A antibodies. Examples of anti-ACTB antibodies suitable for the methods of the disclosure include, but are not limited to, mAbGEa, ACTN05, AC-15, 15G5A11/E2, BA3R, and HHF35.

As described elsewhere herein, libraries of labelling agents can be associated with a particular cell/nucleus feature as well as be used to identify analytes as originating from a particular cell/nucleus population, or sample. Cell or nucleus populations can be incubated with a plurality of libraries such that a cell/nucleus or cells/nuclei include multiple labelling agents. For example, a cell or nucleus can include coupled thereto a lipophilic labeling agent and an antibody. The lipophilic labeling agent can indicate that the cell or nucleus is a member of a particular cell/nucleus sample, whereas the antibody can indicate that the cell/nucleus includes a particular analyte. In this manner, the reporter oligonucleotides and labelling agents can allow multi-analyte, multiplexed analyses to be performed.

In some instances, these reporter oligonucleotides can include nucleic acid barcode sequences that permit identification of the labelling agent which the reporter oligonucleotide is coupled to. The use of oligonucleotides as the reporter can provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents can be achieved through any of a variety of direct or indirect, covalent or non-covalent associations or attachments. For example, oligonucleotides can be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715. Likewise, protein and peptide biotinylation techniques have been developed and are readily available. See, e.g., U.S. Pat. No. 6,265,552. Furthermore, click reaction chemistry such as a Methyltetrazine-PEG5-NHS Ester reaction, a TCO-PEG4-NHS Ester reaction, or the like, can be used to couple reporter oligonucleotides to labelling agents. Commercially available kits, such as those from Thunderlink and Abcam, and techniques common in the art can be used to couple reporter oligonucleotides to labelling agents as appropriate. In another example, a labelling agent is indirectly (e.g., via hybridization) coupled to a reporter oligonucleotide including a barcode sequence that identifies the label agent. For instance, the labelling agent can be directly coupled (e.g., covalently bound) to a hybridization oligonucleotide that includes a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide can be attached to the labeling agent through a labile bond (e.g., chemically labile, photolabile, thermally labile, etc.) as generally described for releasing molecules from supports elsewhere herein. In some instances, the reporter oligonucleotides described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can include a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide can be allowed to hybridize to the reporter oligonucleotide.

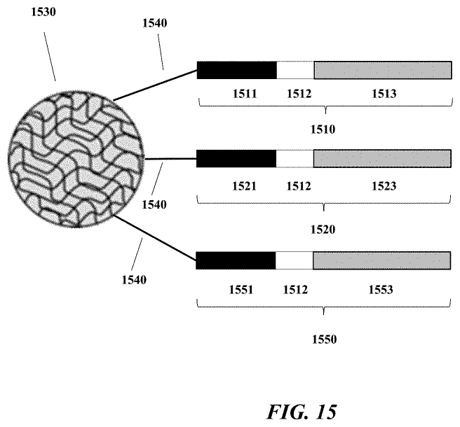

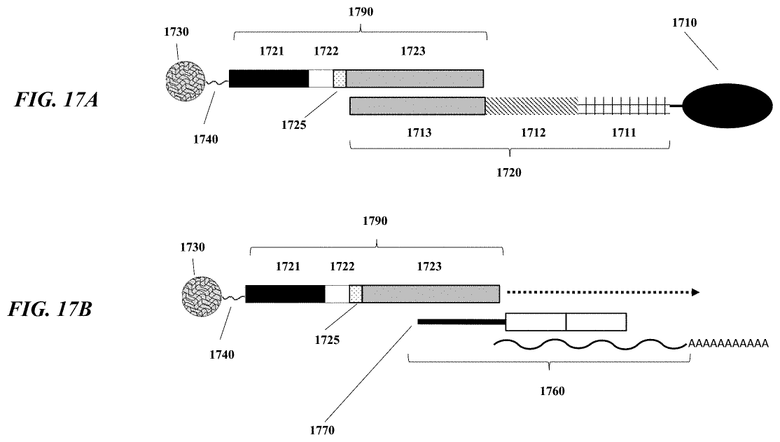

Exemplary barcode molecules attached to a support (e.g., a bead) is shown in FIG. 15. In some embodiments, analysis of multiple analytes (e.g., RNA and one or more analytes using labelling agents described herein) can include nucleic acid barcode molecules as generally depicted in FIG. 15. In some embodiments, nucleic acid barcode molecules 1510 and 1520 are attached to support 1530 via a releasable linkage 1540 (e.g., including a labile bond) as described elsewhere herein. Nucleic acid barcode molecule 1510 can include functional sequence 1511, barcode sequence 1512 and capture sequence 1513. Nucleic acid barcode molecule 1520 can include adapter sequence 1521, barcode sequence 1512, and capture sequence 1523, wherein capture sequence 1523 includes a different sequence than capture sequence 1513. In some instances, adapter 1511 and adapter 1521 include the same sequence. In some instances, adapter 1511 and adapter 1521 include different sequences. Although support 1530 is shown including nucleic acid barcode molecules 1510 and 1520, any suitable number of barcode molecules including common barcode sequence 1512 are contemplated herein. For example, in some embodiments, support 1530 further includes nucleic acid barcode molecule 1550. Nucleic acid barcode molecule 1550 can include adapter sequence 1551, barcode sequence 1512 and capture sequence 1553, wherein capture sequence 1553 includes a different sequence than capture sequence 1513 and 1523. In some instances, nucleic acid barcode molecules (e.g., 1510, 1520, 1550) include one or more additional functional sequences, such as a UMI or other sequences described herein. The nucleic acid barcode molecules 1510, 1520 or 1550 can interact with analytes as described elsewhere herein, for example, as depicted in FIGS. 17A and 17B.

Referring to FIG. 16, in some instances, reporter oligonucleotide 1640 conjugated to an antigen (e.g., 1610, 1620, 1630) can include a functional sequence 1641 (e.g., an adaptor), a barcode sequence that identifies the antigen or antigen-binding molecule (e.g., 1610, 1620, 1630), and functional sequence (e.g., adaptor or capture handle) 1643. Capture handle 1643 can be configured to hybridize to a complementary sequence (e.g., a capture sequence), such as a complementary sequence (e.g., capture sequence) present on a partition-specific barcode molecule (not shown), such as those described elsewhere herein. A capture handle 1643 can include a sequence that is complementary to a capture sequence on a partition-specific barcode molecule. In some instances, a partition-specific barcode molecule is attached to a support (e.g., a bead, such as a gel bead), such as those described elsewhere herein. For example, partition-specific barcode molecules can be attached to the support via a releasable linkage (e.g., comprising a labile bond), such as those described elsewhere herein. In some instances, a reporter oligonucleotide 1640 includes one or more additional functional sequences, such as those described above. In other exemplary embodiments, the partition-specific barcode molecule can include one or more of the following: a peptide tag, an oligonucleotide barcode, a functional sequence, a common barcode, a UMI, and a reporter capture sequence.

In some instances, antigen 1610 is a protein or polypeptide (e.g., an antigen or prospective antigen) conjugated to reporter oligonucleotide 1640. Reporter oligonucleotide 1640 contains a reporter sequence (or reporter barcode sequence) 1642 that identifies protein or polypeptide 1610 and can be used to infer the presence of, e.g., a binding partner of protein or polypeptide 1610 (i.e., a molecule or compound to which the protein or polypeptide binds). In some instances, 1610 is a lipophilic moiety (e.g., cholesterol) comprising reporter oligonucleotide 1640, where the lipophilic moiety is selected such that 1610 integrates into a membrane of a cell or nucleus. Reporter oligonucleotide 1640 contains reporter sequence 1642 that identifies lipophilic moiety 1610 which in some instances is used to tag cells (e.g., groups of cells, cell samples, etc.) for multiplex analyses as described elsewhere herein.

In some instances, the antigen-binding molecule is an antibody 1620 (or an epitope binding fragment thereof) including reporter oligonucleotide 1640. Reporter oligonucleotide 1640 includes reporter sequence 1642 that identifies antibody 1620 and can be used to infer the presence of, e.g., a target of antibody 1620 (i.e., a molecule or compound to which antibody 1620 binds).

In some embodiments, the agent to be labeled 1630 includes an MHC molecule 1631 including peptide 1632 and oligonucleotide 1640 that identifies peptide 1632. In some instances, the MHC molecule is coupled to a support 1633. In some instances, support 1633 is streptavidin (e.g., MHC molecule 1631 can include biotin). In some embodiments, support 1633 is a polysaccharide, such as dextran. In some instances, reporter oligonucleotide 1640 can be directly or indirectly coupled to MHC labelling agent 1630 in any suitable manner, such as to MHC molecule 1631, support 1633, or peptide 1632. In some embodiments, labelling agent 1630 includes a plurality of MHC molecules, e.g., is an MHC multimer, which can be coupled to a support (e.g., 1633). There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labeling of various antigens, including antibody and MHC-based labelling agents, reporter oligonucleotides, and methods of use, see, e.g., U.S. Pat. No. 10,550,429 and U.S. Pat. Pub. 20190367969.

Referring to FIG. 17A, in an instance where cells are labelled with labeling agents, capture sequence 1723 can be complementary to an adapter sequence of a reporter oligonucleotide. Cells can be contacted with one or more reporter oligonucleotide 1720 conjugated labelling agents 1710 (e.g., polypeptide, antibody, or others described elsewhere herein). In some cases, the cells can be further processed prior to barcoding. For example, such processing steps can include one or more washing and/or cell sorting steps. In some instances, a cell that is bound to labelling agent 1710 which is conjugated to oligonucleotide 1720 and support 1730 (e.g., a bead, such as a gel bead) including nucleic acid barcode molecule 1790 is partitioned into a partition amongst a plurality of partitions (e.g., a droplet of a droplet emulsion or a well of a microwell array). In some instances, the partition includes at most a single cell bound to labelling agent 1710. In some instances, reporter oligonucleotide 1720 conjugated to labelling agent 1710 (e.g., polypeptide, an antibody, pMHC molecule such as an MHC multimer, etc.) includes a first functional sequence 1711 (e.g., a primer sequence), a barcode sequence 1712 that identifies the labelling agent 1710 (e.g., the polypeptide, antibody, or peptide of a pMHC molecule or complex), and a capture handle sequence 1713. Capture handle sequence 1713 can be configured to hybridize to a complementary sequence, such as capture sequence 1723 present on a nucleic acid barcode molecule 1790 (e.g., partition-specific barcode molecule). In some instances, oligonucleotide 1720 includes one or more additional functional sequences, such as those described elsewhere herein.

Barcoded nucleic acid molecules can be generated (e.g., via a nucleic acid reaction, such as nucleic acid extension or ligation) from the constructs described in FIGS. 17A-17B. For example, capture handle sequence 1713 can then be hybridized to complementary capture sequence 1723 to generate (e.g., via a nucleic acid reaction, such as nucleic acid extension or ligation) a barcoded nucleic acid molecule including cell barcode (e.g., common barcode or partition-specific barcode) sequence 1722 (or a reverse complement thereof) and reporter sequence 1712 (or a reverse complement thereof). In some embodiments, the nucleic acid barcode molecule 1790 (e.g., partition-specific barcode molecule) further includes a UMI (1725). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808, which is incorporated herein by reference in its entirety. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) can be performed. For example, the workflow can include a workflow as generally depicted in any of FIGS. 17A-17B, or a combination of workflows for an individual analyte, as described elsewhere herein. For example, by using a combination of the workflows as generally depicted in FIGS. 17A-17B, multiple analytes can be analyzed.

In some instances, analysis of an analyte (e.g., a nucleic acid, a polypeptide, a carbohydrate, a lipid, etc.) includes a workflow as generally depicted in FIG. 17A. A nucleic acid barcode molecule 1790 can be co-partitioned with the one or more analytes. In some instances, nucleic acid barcode molecule 1790 is attached to a support 1730 (e.g., a bead, such as a gel bead), such as those described elsewhere herein. For example, nucleic acid barcode molecule 1790 can be attached to support 1730 via a releasable linkage 1740 (e.g., including a labile bond), such as those described elsewhere herein. Nucleic acid barcode molecule 1790 can include a functional sequence 1721 and optionally include other additional sequences, for example, a barcode sequence 1722 (e.g., common barcode, partition-specific barcode, or other functional sequences described elsewhere herein), and/or a UMI sequence 1725. The nucleic acid barcode molecule 1790 can include a capture sequence 1723 that can be complementary to another nucleic acid sequence, such that it can hybridize to a particular sequence.

For example, capture sequence 1723 can include a sequence configured to hybridize to a nucleic acid probe or probes as described herein. Referring to FIG. 17B, nucleic acid barcode molecule 1790 includes capture sequence 1723 complementary to a sequence of one or more probes (e.g., a pair of probes) hybridized to an RNA molecule 1760 from a biological particle (e.g., a cell or a nucleus). The probes may be ligated to one another and optionally hybridized to the RNA molecule for the barcoding process. Capture sequence 1723 can include a known or targeted sequence or a random sequence. The capture sequence 1723 can include a sequence that is complementary to a probe sequence of a barcode sequence 1770 that is part of a probe. In some instances, a nucleic acid extension reaction can be performed, thereby generating a barcoded nucleic acid product including capture sequence 1723, the functional sequence 1721, UMI sequence 1725, any other functional sequence, and a sequence corresponding to the probes depicted as part of 1760. In this embodiment, the resulting barcoded nucleic acid product would not include the original sequence of an RNA molecule but rather the sequence of the probes hybridized to the RNA molecule. The barcoded nucleic acid product lacks the original RNA molecule.

Additional methods and compositions suitable for barcoding nucleic acid molecules are described in U.S. Patent Publication Nos. 2015/0376609, 2019/0367969, US 2020-0239874, US 2021-0040551, as well as International PCT applications WO2019/165318 and PCT/US20/48620.

Embodiments of the invention, which are not meant to be limiting, are described in the numbered paragraphs below.

1. A method of extracting and isolating fixed cells and nuclei from a frozen biological issue, comprising the steps of:

providing a sample of the frozen biological tissue;

treating the sample of biological tissue with an organic fixing agent;

separating the biological tissue into tissue segments to facilitate perfusion and fixation of the biological tissue by the organic fixing agent;

quenching the fixation of the tissue segments;

treating the fixed tissue segments with a dissociation enzyme mixture;

pulverizing the fixed tissue segments into dissociated tissue particles;

adding a first buffer solution to the dissociated tissue particles to form a mixture;

centrifuging the mixture to yield at least one pellet;

adding the at least one pellet to a second buffer solution to form a suspension; and

filtering the suspension to yield a plurality of fixed, isolated cells and nuclei.

2. The method of paragraph 1, wherein the frozen biological tissue is snap frozen.

3. The method of paragraph 1, wherein the organic fixing agent is selected from the group consisting of an alcohol, ketone, aldehyde, cross-linking agent, disuccinimidyl suberate (DSS), dimethylsuberimidate (DMS), formalin, dimethyladipimidate (DMA), dithio-bis(-succinimidyl propionate) (DSP), disuccinimidyl tartrate (DST), ethylene glycol bis (succinimidyl succinate) (EGS), bis-imidazole-carboxylate compounds, and combinations thereof.

4. The method of paragraph 3, wherein the organic fixing agent comprises formalin.

5. The method of paragraph 4, wherein the formalin comprises about 2% to about 6% by weight formaldehyde in water.

6. The method of paragraph 1, wherein the treating with an organic fixing agent comprises applying the organic fixing agent to the biological tissue at a temperature of about zero to about 5 degrees Celsius.

7. The method of paragraph 1, wherein the treating step comprises placing the organic fixing agent over a bed of ice and immersing the sample of biological tissue in the organic fixing agent.

8. The method of paragraph 1, further comprising the step of separating the segments of biological tissue into smaller segments at one or more periodic intervals during treatment with the organic fixing agent, to further facilitate perfusion and fixation of the biological tissue.

9. The method of paragraph 7, wherein the segments of biological tissue are separated into successively smaller segments at two or more periodic intervals.

10. The method of paragraph 7, wherein the segments of biological tissue are separated into successively smaller segments at three or more periodic intervals.

11. The method of paragraph 7, wherein the segments of biological tissue are separated into successively smaller segments at four or more periodic intervals.

12. The method of paragraph 1, wherein the sample of biological tissue is treated with the organic fixing agent for a time of about 10 to about 30 minutes.

13. The method of paragraph 1, wherein the sample of biological tissue is treated with the organic fixing agent for a time of about 15 to about 25 minutes.

14. The method of paragraph 8, wherein a sum of the one or more periodic intervals is about 10 to about 30 minutes.

15. The method of paragraph 8, wherein a sum of the one or more periodic intervals is about 15 to about 25 minutes.

16. The method of paragraph 8, wherein each of the one or more periodic intervals is about 1 to about 10 minutes.

17. The method of paragraph 8, wherein each of the one or more periodic intervals is about 3 to about 7 minutes.

18. The method of paragraph 1, wherein the step of quenching the fixation of the tissue segments comprises the step of immersing the fixed tissue segments in a quenching medium.

19. The method of paragraph 18, wherein the quenching medium comprises a phosphate buffering solution.

20. The method of paragraph 19, wherein the phosphate buffering solution comprises 1×PBS.

21. The method of paragraph 19, wherein the phosphate buffer solution further comprises a fetal bovine serum.

22. The method of paragraph 21, wherein the fetal bovine serum is present at about 5% to about 15% by weight of the phosphate buffer solution.

23. The method of paragraph 18, wherein the quenching medium comprises about 55% to about 85% by weight ethanol and about 15% to about 35% by weight water.

24. The method of paragraph 18, wherein the quenching medium has a temperature of about zero to about 10 degrees Celsius.

25. The method of paragraph 1, wherein the dissociation enzyme mixture comprises collagenase and dithiothreitol.

26. The method of paragraph 25, wherein the dissociation enzyme mixture further comprises a ribonuclease inhibitor.

27. The method of paragraph 1, wherein the pulverizing step comprises passing the fixed tissue segments through a strainer.

28. The method of paragraph 27, wherein the strainer has openings of about 5 microns to about 200 microns in size.

29. The method of paragraph 27, wherein the strainer has openings of about 25 microns to about 100 microns in size.

30. The method of paragraph 1, wherein the first buffer solution comprises a phosphate buffer saline solution.

31. The method of paragraph 30, wherein the first buffer solution further comprises bovine serum albumin.

32. The method of paragraph 1, wherein the centrifuging is performed at a temperature of about zero to about 10 degrees Celsius.

33. The method of paragraph 1, wherein the centrifuging is performed at a force of about 50 g to about 2500 g.

34. The method of paragraph 1, wherein the centrifuging is performed at a force of about 200 g to about 1000 g.

35. The method of paragraph 1, wherein the second buffer solution comprises a phosphate buffer saline solution.

36. The method of paragraph 35, wherein the second buffer solution further comprises bovine serum albumin.

37. The method of paragraph 1, wherein the filtering step comprises passing the suspension through a filter having a median pore size of about 1 micron to about 500 microns.

38. The method of paragraph 1, wherein the filtering step comprises passing the suspension through a filter having a median pore size of about 10 microns to about 100 microns.

39. A method of extracting and isolating fixed cells and nuclei from a frozen biological tissue, comprising the steps of:

fixing a plurality of tissue segments derived from the frozen biological tissue with a fixing agent;

quenching the fixation of the tissue segments;

treating the fixed tissue segments with a dissociation enzyme;

separating the fixed tissue segments into dissociated tissue particles;

adding a first buffer solution to the dissociated tissue particles to form a mixture;

centrifuging the mixture to yield at least one pellet;

adding the at least one pellet to a second buffer solution to form a suspension; and

filtering the suspension to yield a plurality of fixed, isolated cells and nuclei.

40. The method of paragraph 39, wherein quenching the fixation comprises exposing the fixed tissue segments to a mixture of phosphate buffered saline solution and fetal bovine serum.

41. The method of paragraph 40, wherein the mixture has a temperature of about zero to about 10 degrees Celsius.

42. The method of paragraph 39, wherein the frozen biological tissue is snap frozen.

43. The method of paragraph 39, wherein the fixing agent comprises an organic fixing agent.

44. The method of paragraph 43, wherein the organic fixing agent comprises formaldehyde.

45. The method of paragraph 39, wherein the dissociation enzyme mixture comprises collagenase and dithiothreitol.

46. The method of paragraph 45, wherein the dissociation enzyme mixture further comprises a ribonuclease inhibitor.

47. The method of paragraph 1, wherein the separating step comprises forcing the fixed tissue segments through a strainer.

48. The method of paragraph 47, wherein the strainer has openings of about 5 microns to about 200 microns in diameter.

49. The method of paragraph 39, wherein the first buffer solution comprises a phosphate buffer saline solution.

50. The method of paragraph 49, wherein the first buffer solution further comprises bovine serum albumin.

51. The method of paragraph 39, wherein the centrifuging is performed at a temperature of about zero to about 10 degrees Celsius.

52. The method of paragraph 51, wherein the centrifuging is performed at a force of about 50 g to about 2500 g.

53. The method of paragraph 39, wherein the second buffer solution comprises a phosphate buffer saline solution.

54. The method of paragraph 53, wherein the second buffer solution further comprises bovine serum albumin.

55. The method of paragraph 39, wherein the filtering step comprises passing the suspension through a filter having a median pore size of about 1 micron to about 200 microns.

56. A method of extracting and isolating fixed cells and nuclei from a frozen biological issue, comprising the steps of:

providing a sample of snap-frozen biological tissue;

treating the biological tissue with an organic fixing agent;

separating the biological tissue into tissue segments to facilitate perfusion and fixation of the biological tissue by the organic fixing agent;

dissociating the fixed tissue segments into dissociated tissue particles;

adding a first buffer solution to the dissociated tissue particles to form a mixture;

centrifuging the mixture to yield at least one pellet;

adding the at least one pellet to a second buffer solution to form a suspension; and

filtering the suspension to yield a plurality of fixed, isolated cells and nuclei.

57. The method of paragraph 56, wherein the step of treating the biological tissue with an organic fixing agent comprises exposing the snap-frozen biological tissue to a chilled organic fixing agent at a temperature of about zero to about 10 degrees Celsius.

58. The method of paragraph 56, wherein the step of separating the biological tissue into tissue segments is performed before and/or during the step of treating the biological tissue with the organic fixing agent.

59. The method of paragraph 56, wherein the step of dissociating the fixed tissue segments into tissue particles comprises the steps of:

treating the fixed tissue segments with a dissociation enzyme mixture; and

pulverizing the fixed tissue segments into dissociated tissue particles.

60. The method of paragraph 59, wherein the dissociation enzyme mixture comprises collagenase and dithiothreitol.

61. The method of paragraph 60, wherein the dissociation enzyme mixture further comprises a ribonuclease inhibitor.

62. The method of paragraph 59, wherein the pulverizing step comprises passing the fixed tissue segments through a strainer.

63. The method of paragraph 62, wherein the strainer has openings of about 5 microns to about 200 microns in diameter.

64. The method of paragraph 56, wherein the first buffer solution comprises a phosphate buffer saline solution.

65. The method of paragraph 63, wherein the first buffer solution further comprises bovine serum albumin.

66. The method of paragraph 56, wherein the centrifuging is performed at a temperature of about zero to about 10 degrees Celsius.

67. The method of paragraph 66, wherein the centrifuging is performed at a force of about 50 g to about 2500 g.

68. The method of paragraph 56, wherein the second buffer solution comprises a phosphate buffer saline solution.

69. The method of paragraph 68, wherein the second buffer solution further comprises bovine serum albumin.

70. The method of paragraph 56, wherein the filtering step comprises passing the suspension through a filter having a median pore size of about 1 micron to about 200 microns.

71. A method of ribonucleic acid (RNA) analysis in biological tissue samples comprising

a) contacting a biological tissue sample with an organic fixing agent;

b) in the presence of the organic fixing agent, dividing the biological tissue sample into tissue segments to allow perfusion of the organic fixing agent into the tissue segments;

c) dissociating the tissue segments to provide a plurality of single cells and/or single nuclei; and

d) performing RNA analysis on the plurality of single cells and/or single nuclei.

72. The method of paragraph 71, wherein the biological tissue of step (a) is frozen or fresh biological tissue.

73. The method of paragraph 71 or 72, wherein the RNA analysis comprises RNA templated ligation.

74. The method of paragraph 73, wherein the RNA template ligation comprises detecting ligation products in the plurality of single cells and/or single nuclei.

75. The method of paragraph 73, wherein the plurality of single cells and/or single nuclei are partitioned into a plurality of partitions.

76. The method of paragraph 75, wherein the plurality of partitions comprises a plurality of ligations products.

77. The method of paragraph 74, wherein the plurality of single cells and/or single nuclei are partitioned into a plurality of partitions following detection of the ligation products.

78. The method of paragraph 75, wherein the plurality of partitions is a plurality of droplets or a plurality of microwells.

79. The method of paragraph 76, further comprising generating a plurality of barcoded nucleic acid molecules, wherein a barcoded nucleic acid molecule of the plurality of barcoded molecules comprises a partition-specific sequence and a sequence corresponding to the plurality of ligation products.

80. The method of any one of paragraphs 71-79, wherein the steps are performed in the absence of an un-fixing reagent.

81. A composition, comprising the fixed cells and nuclei from any one of paragraphs 1-70.

### Examples

The following examples are for illustrating various embodiments and are not to be construed as limitations.

### Example 1. Processing Tissue to Obtain Single Cells

A sample of snap frozen human lymph node tissue having the approximate size of a corn kernel was removed from liquid nitrogen storage and placed directly into a tube containing a chilled 4% formaldehyde solution on ice. After 5 minutes, the tissue sample was cut into four smaller segments while immersed in the formaldehyde solution. After another 5 minutes, the tissue segments were cut into smaller segments while immersed, and this process was repeated after another 5 minutes. The periodic segmenting of the tissue segments during immersion facilitated perfusion of the formaldehyde solution into the tissue segments. After 20 minutes of fixing, the tissue segments were removed from the tube and placed into a chilled solution of 1×PBS with 10% FBS to quench the fixation reactions.

The fixed and quenched tissue segments were removed from the quenching medium and placed in a dissociation enzyme mixture containing 0.1 mg/ml Liberase (Collagenase I and II) in RPMI 1640, 10 mM DTT, and 0.2 units/μl ribonuclease inhibitor from Thermo Fisher Scientific. The tissue segments were incubated in the dissociation enzyme mixture for 10 minutes at 370 C with intermittent trituration to further dissociate the tissue segments. The fixed tissue segments were then removed from the dissociation enzyme mixture and were mashed through a 70 μM Miltenyi MACs cell strainer using the back of a syringe plunger and the resulting tissue particles were collected in a new tube. The strainer was then washed using a chilled buffer solution of 1×PBS with 0.04% by weight BSA and the effluent was collected in the new tube.

The mixture of tissue particles and chilled buffer solution effluent was centrifuged at 500 g force for 5 minutes at 40 C. The resulting pellet was collected and re-suspended in a buffer solution of 1×PBS with 0.04% by weight BSA. The resulting suspension was filtered using a 40 μM FlowMi filter to yield a collection of fixed, isolated tissue cells and cell nuclei.

### Example 2. RNA-Templated Ligation Using Single Cells from Tissue

The collection of fixed, isolated tissue cells and cell nuclei was processed following the RNA-templated ligation protocol set forth below. The objective was to produce sequence-able libraries for targeted single cell RNA sequencing by first hybridizing targeted probes to the template RNA of the cells and nuclei, followed by ligation. The ligated cells were then taken through GEM gene generation, followed by further processing to produce libraries for sequencing (e.g., on an Illumina instrument).

To perform RNA-template ligation, 900 μl of Hybridization Buffer as shown in Table 1, was added to 100 μl stained, fixed PBMCs. This was incubated at 45° C. for 2 hours with shaking at 500 rpm. The mixture was then transferred to a 15 ml screw-capped tube and Post-Hybridization Buffer (Table 2) was added to 10 ml, and inverted slowly 5-times. The tubes were centrifuged at 500×g for 5 minutes at room temperature. The supernatant was removed without disturbing the pellet. The pellet was gently resuspended using a wide-bore pipette tip in 1 ml of Post-Hybridization Buffer. The mixture was then brought to 10 ml with Post-Hybridization Buffer at 37° C. The tube was centrifuged again, supernatant removed as above, and the pellet was gently resuspended in 1 ml Ligation Buffer (Table 3). The mixture was then brought to 5 ml with Ligation Buffer at 37° C. the tube was centrifuged again, supernatant removed as above, and pellet resuspended in 200 μl Ligation Buffer at 37° C. RNL2 at 10 μl was added. The mixture was incubated for 2 hours at 37° C. with shaking (500 rpm). The tubes were centrifuged, supernatant removed as above, and pellet resuspended in 200 μl Cell Resuspension Buffer (Table 4).

TABLE 1
Hybridization Buffer

| | Stock | Final | Amount |
|---|---|---|---|
| Ingredient | Concentration | Concentration | Added (ml) |
| SSC Buffer | 20x | 2x | 0.010 |
| Formamide | 100% | 20% | 0.020 |
| BSA | 10% | 0.2% | 0.002 |
| RNase Inhibitor | 40 U/μl | 0.2 U/μl | 0.001 |
| Yeast tRNA | 10 mg/ml | 0.1 mg/ml | 0.001 |
| Dextran Sulfate | 50% | 10% | 0.020 |
| LHS + RHS Pooled | 10 μM | 1 μM | 0.010 |
| Probes | | | |
| Water | | | 0.037 |
| | | Total (ml) | 0.100 |

TABLE 2
Post-Hybridization Buffer

| | Stock | Final | Amount |
|---|---|---|---|
| Ingredient | Concentration | Concentration | Added (ml) |
| SSC Buffer | 20x | 2x | 4.000 |
| RNase Inhibitor | 40 U/μl | 0.2 U/μl | 0.200 |
| BSA | 10% | 0.1% | 0.400 |
| Yeast tRNA | 10 mg/ml | 0.1 mg/ml | 0.400 |
| Water | | | 35.000 |
| | | Total (ml) | 40.000 |

TABLE 3
Ligation Buffer

| | Stock | Final | Amount |
|---|---|---|---|
| Ingredient | Concentration | Concentration | Added (ml) |
| 1 M Tris, pH 7.5 | 1000 mM | 50 mM | 0.600 |
| MgCl₂ | 2000 mM | 10 mM | 0.060 |
| DTT | 1000 mM | 5 mM | 0.060 |
| ATP | 10 mM | 0.5 mM | 0.600 |
| RNase Inhibitor | 40 U/μl | 0.2 U/μl | 0.060 |
| Water | | | 10.620 |
| | | Total (ml) | 12.000 |

TABLE 4
Cell Resuspension Buffer

| | Stock | Final | Amount |
|---|---|---|---|
| Ingredient | Concentration | Concentration | Added (ml) |
| PBS | 1x | 1x | 4.980 |
| BSA | 10% mM | 0.04% | 0.020 |
| | | Total (ml) | 5.000 |

Following hybridization and ligation as above, the fixed cells and nuclei were run using a modification of the Single Cell 3′ Reagent Kit, Version 3, available from 10× Genomics. Modifications include the following GEM Generation Master Mix having Bst 2.0 WarmStart® DNA Polymerase from New England BioLabs (Table 5) and the following GEM RT thermal cycler incubation (Table 6).

TABLE 5
Modified GEM Generation Master Mix

| | Stock | Concentration | 1 Library | 16 Libraries |
|---|---|---|---|---|
| Ingredient | Concentration | in GEM | (volume in μl) | (volume in μl) |
| 10x BST Iso 2 | 10x | 1x | 9.8 | 173.2 |
| Buffer | | | | |
| Reducing Agent | 1000 mM | 10 mM | 1.0 | 17.3 |
| B | | | | |
| Glycerol | 50% | 7% | 11.5 | 202.8 |
| dNTP Mix | 10 mM | 0.5 mM | 4.9 | 86.6 |
| Bst 2.0 | 120 U/μl | 0.5 U/μl | 0.4 | 7.2 |
| Polymerase | | | | |
| Synperonic | 10.0% | 0.5% | 4.9 | 86.6 |
| Water | | | 7.4 | 130.3 |
| | | Total Master Mix | 40.0 μl | 704.0 μl |
| | | added to sample (μl) | | |

TABLE 6
GEM RT Thermal Cycler Incubation

| Step | Temperature | Time |
|---|---|---|
| Polymerase Extension | 55° C. | 45 min |
| Heat-Kill | 80° C. | 20 min |
| Hold | 4° C. | ∞ |

After GEM generation, further processing was done following the Single Cell 3′ Reagent Kits v3 User Guide, published by 10× Genomics. Another modification was done during the Post GEM-RT Cleanup step. After removing the 125 μl Recovery Agent/partitioning oil (pink) from each sample, 60 μl of the aqueous phase (clear) is left. The 60 μl was split into two identical polymerase chain (PCR) reactions where each 30 μl was added to two separate tubes containing 70 μl of the following master mix containing a partial Illumina Nextera read 1 primer sequence and partial Illumina Small RNA sequencing primer read 2 (Table 7).

TABLE 7
Master Mix 2

| | Stock | Final | 1 Library | 16 Libraries |
|---|---|---|---|---|
| Ingredient | Concentration | Concentration | (volume in μl) | (volume in μl) |
| Qiagen Mix | 2x | 1x | 50 | 880.0 |
| ART349 (Nextera | 100 μM | 1 μM | 1 | 17.6 |
| Read 1) | | | | |
| ART383 (Small | 100 μM | 1 μM | 1 | 17.6 |
| RNA Read 2) | | | | |
| Water | | | 18 | 316.8 |
| Sample | | | 30 | — |
| | | Total Volume (μl) | 100 μl | 1232 μl |
| | | Total Master Mix | 70 μl | |
| | | added to sample | | |
| | | (μl) | | |

The 100 μl PCR reactions had the thermal cycler incubation parameters shown in Table 8.

TABLE 8
Thermal Cycler Incubation

| | | | |
|---|---|---|---|
| Initial Denaturation | 98° C. | 3 min | 1x |
| Denaturation | 98° C. | 15 sec | |
| Annealing | 63° C. | 20 sec | 13 |
| Extension | 72° C. | 1 min | |
| Final Extension | 72° C. | 1 min | 1x |
| Hold | 4° C. | ∞ | ∞ |

After the first PCR reaction, cDNA Cleanup with solid phase reverse immobilization beads (SPRiselect®) was done following the Single Cell 3′ Reagent Kits v3 User Guide. Instead of a 0.6×SPRI cleanup, a 1.8×SPRI cleanup was done where 180 μl of SPRiselect® reagent was added to each sample. Another modification was the final elution volume of Buffer EB (elution buffer) which was 25 μl for each identical sample and then combined for a total of 50 μl of eluted cDNA instead of the 40 μl in the Single Cell 3′ Reagent Kits v3 User Guide.

Ten μl of the amplified RNA-Templated Ligation cDNA was then taken into a Sample Index PCR with the master mix shown in Table 9 containing the P5-partial Nextera read 1 and P7-small RNA sample index primers used in Illumina bridge amplification.

TABLE 9
Master Mix 3

| | Stock | Final | 1 Library | 8 Libraries |
|---|---|---|---|---|
| Ingredient | Concentration | Concentration | (volume in μl) | (volume in μl) |
| P5-NexteraR1 | 200 μM | 1 μM | 0.5 | 4.4 |
| Amp Mix | 2x | 1x | 50.0 | 440.0 |
| (2000047) | | | | |
| Water | | | 34.5 | 303.6 |
| MSxx smallRNA | 20 μM | 1 μM | 5 | — |
| SI Index | | | | |
| Amplified | | | 10 | — |
| RNA-Templated | | | | |
| Ligation | | | | |
| | | Total Volume (μl) | 100 μl | 748 μl |
| | | Total Master Mix, | 85 μl | |
| | | added to sample (μl) | | |

Thermal cycler parameters for the 100 μl sample index PCR were as shown in Table 10.

TABLE 10
Thermal Cycler Incubation

| | | | |
|---|---|---|---|
| Initial Denaturation | 98° C. | 35 sec | 1x |
| Denaturation | 98° C. | 20 sec | |
| Annealing | 60° C. | 30 sec | 14 |
| Extension | 72° C. | 20 sec | |
| Final Extension | 72° C. | 1 min | 1x |
| Hold | 4° C. | ∞ | ∞ |

After the sample index PCR reaction, cDNA Cleanup with SPRIselect® was done following the Single Cell 3′ Reagent Kits v3 User Guide. Instead of a 0.6×SPRI cleanup, a 1.2×SPRI cleanup was done where 120 μl of SPRIselect® reagent was added to each sample. The Final elution volume of Buffer EB (elution buffer) was 40 μl as stated in the Single Cell 3′ Reagent Kits v3 User Guide.

### Example 3. Analysis

The resulting final sequenceable libraries from Example 2 were diluted 1:10 in buffer EN and were analyzed using an Agilent 2100 Bioanalyzer. The bioanalyzer provides a microfluidics-based platform for sizing, quantification and quality control of DNA and RNA. After loading the sample of fixed, isolated cells and cell nuclei on the desired chip, the sample moves through microchannels and sample components are electrophilically separated. Smaller components migrate faster than larger ones. Fluorescent dye molecules intercalate into the DNA and RNA strands, which can then be detected by their fluorescence and translated into gel-like images (bands) and electropherograms (peaks).

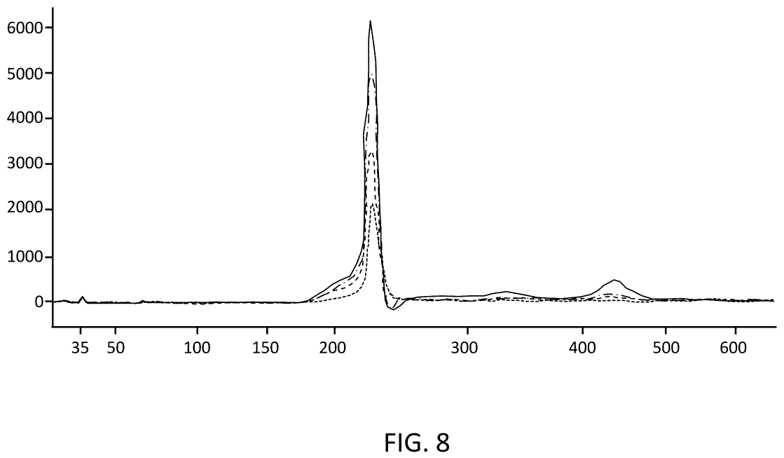

FIG. 8, representing the invention, is a bioanalyzer plot of showing the ligation products resulting from RNA-templated ligation of the collection of fixed, isolated cells and cell nuclei prepared according to the method of the invention. A PL Minus Strand (SC3P 50 bp R2) was used for the index scheme. Four large peaks, representing four cDNA ligation products of four different cell loads of 1000, 2000, 3000 and 6000 cells, appeared at about 230 bae pairs (bp) with intensities ranging from 2200 fluorescent units (for the 1000-cell load) to 6200 fluorescent units (for the 6000-cell load). These large peaks occurring at approximately 230 bp indicate significant amounts of ligation product.

When this instrument was used to analyze ligation products resulting from RNA-templated ligation of the collection of fixed, isolated cells and cell nuclei prepared according to the method of the invention, four large peaks, representing four cDNA ligation products of four different cell loads of 1000, 2000, 3000 and 6000 cells, appeared at about 230 base pairs (bp) with intensities ranging from 2200 fluorescent units (for the 1000-cell load) to 6200 fluorescent units (for the 6000-cell load). These large peaks occurring at approximately 230 bp indicate significant amounts of ligation product.

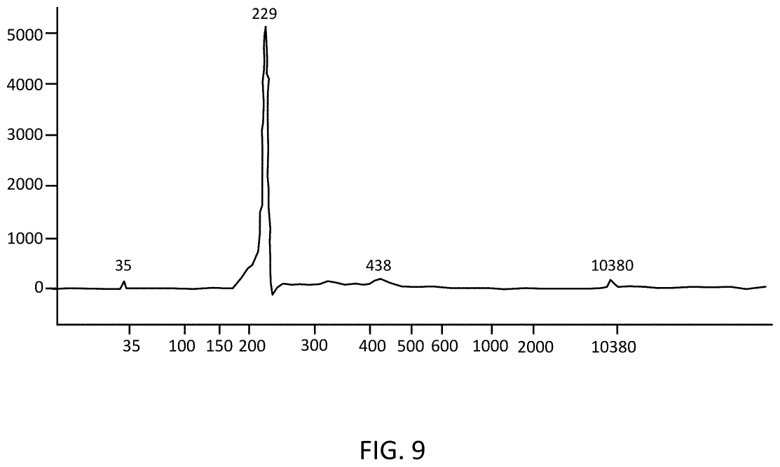

FIG. 9 is a replicate of the 3000-cell load electropherogram alone with the bp size highlighted for the ligation products at 230 bp. The 35 bp peak is the lower marker and the 10380 bp peak is the upper marker. All of the other peaks represent primer dimers and PCR artifacts.

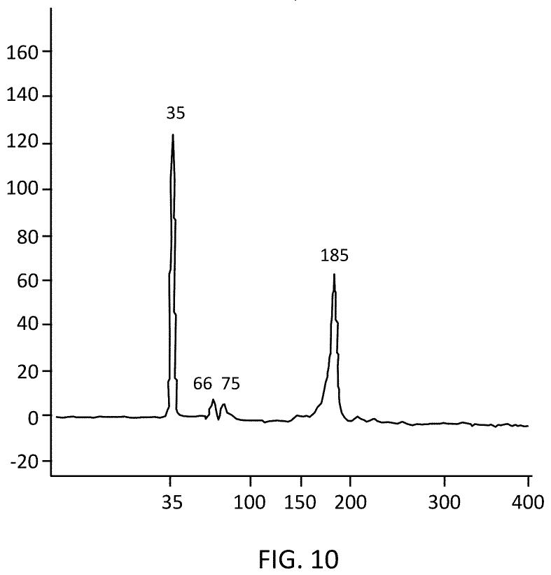

FIG. 10, representing a control, is a bioanalyzer plot showing the products resulting from RNA-templated ligation using the same ligation protocol, where the snap-frozen human lymph node tissue sample was prepared and pulverized using conventional techniques. The snap-frozen tissue sample was first treated with a chilled (4° C.) NP40 based lysis buffer composed of 10 mM Tris-HCl, 10 mM NaCl, 3 mM MgCl2, and 0.1% Nonidet™ P40 Substitute in Nuclease-Free Water. The tissue was then pulverized using mechanical douncing. The resulting pulverized tissue particles were centrifuged, re-suspended and washed using PBS with 1% BSA and 0.2 units/ul ribonuclease inhibitor. The washed tissue particles were then fixed using 4% formaldehyde, unlike the invention, where the formaldehyde fixing is accomplished before further processing. The resulting suspension was then filtered using a 40 μM FlowMi filter, and the dissociated tissue nuclei were then subjected to the RNA Templated Ligation Protocol V1.

In another experiment, not using the single-cell preparation methods disclosed here but, instead, using a standard method, a snap-frozen human lymph node tissue sample was prepared and pulverized using conventional techniques. The snap-frozen tissue sample was first treated with a chilled (4° C.) NP40 based lysis buffer composed of 10 mM Tris-HCl, 10 mM NaCl, 3 mM MgCl2, and 0.1% Nonidet™ P40 Substitute in Nuclease-Free Water. The tissue was then pulverized using mechanical douncing. The resulting pulverized tissue particles were centrifuged, re-suspended and washed using PBS with 1% BSA and 0.2 units/ul ribonuclease inhibitor. The washed tissue particles were then fixed using 4% formaldehyde, unlike the invention, where the formaldehyde fixing is accomplished before further processing. The resulting suspension was then filtered using a 40 μM FlowMi filter, and the dissociated tissue nuclei were then subjected to the RNA Templated Ligation Protocol. When the ligation products were analyzed using the Agilent 2100 Bioanalyzer, no products at −230 bp were detected. Only a small peak at 185 bp was detected, which likely represented primer-dimers or PCR artifacts.

Additionally, sequencing results from the methods of the present invention not only confirmed a high number of ligation products, but also a high fraction of reads in cells. Fraction of reads in cells is a metric that measures background and is indicative of cell health. A low fraction of reads in cells indicates poor cell health and an increase in ambient RNA content during GEM generation. A high fraction of reads in cells indicates a high number of reads that have valid barcode, UMI, map confidently to the transcriptome, and are in cell associated partitions. The primary metrics were as shown in Table 11.

TABLE 11
Sequencing Metrics

| Percent raw reads with | Percent raw reads unambiguously | Percent raw reads on- |
|---|---|---|
| some on-target mapping: 96.5% | mapped: 96.2% | target and unambiguously |
| | | mapped: 96.1% |
| Number of on-target reads: | Number of unambiguously | Number of unambiguously |
| 26,116,470 | mapped reads: 26,030,459 | mapped on-target reads: |
| | | 23,066,548 |
| Median panel reads per cell: 5,153 | Median panel UMI's per cell: | Median panel genes per |
| | 1,346 | cell: 299 |
| Mean raw reads per cell: 6,205 | Mean raw panel reads per cell: | Mean raw non-panel reads |
| | 6,054 | per cell: 150 |

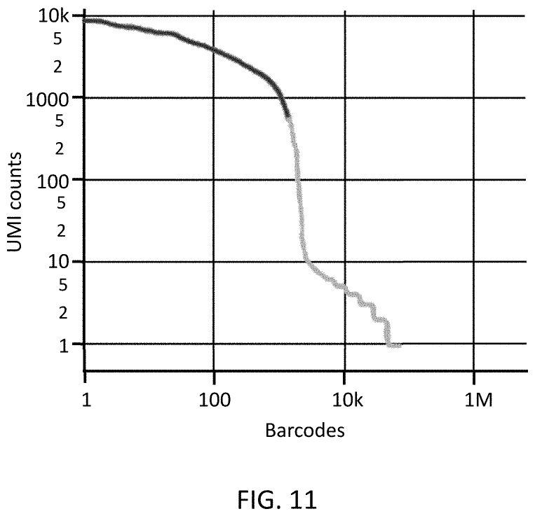

The barcode rank plot shown in FIG. 11 shows the distribution of barcode counts and which barcodes were inferred to be associated with cells. The y-axis is the number of UMI counts mapped to each barcode and the x-axis is the number of barcodes below that value. A steep drop-off is indicative of good separation between the cell-associated barcodes and the barcodes associated with empty partitions. The barcode rank plot also shows a good separation between signal and noise. The singular ‘knee’ behavior indicates there is a clear separation between our cell-associated barcodes of interest and background of non-cell-associated barcodes.

A barcode rank plot (number of UMI counts mapped to each barcode vs. number of barcodes below that value) showed the distribution of barcode counts and which barcodes were inferred to be associated with cells. The barcode rank plot indicated good separation between the cell-associated barcodes and the barcodes associated with empty partitions. The barcode rank plot also showed good separation between signal and noise. The plot showed a singular “knee,” indicating a clear separation between the cell-associated barcodes of interest and background of non-cell-associated barcodes. Some of the data are shown in Table 12.

TABLE 12
Barcode Data

| | |
|---|---|
| Estimated | 1,196 |
| number of cells | |
| Fraction reads | 89.0% |
| in cells | |
| Mean reads per | 7,294 |
| cell | |
| Median genes | 298 |
| per cell | |
| Total genes | 1,661 |
| detected | |
| Median UMI | 1,350 |
| counts per cell | |

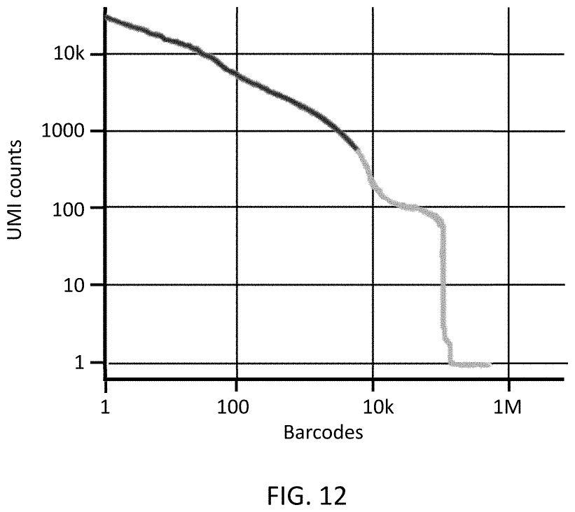

By comparison, the unbiased gene expression patterns for the unfixed nuclei were not as good in terms of cleanliness of the library due to the lower fraction of reads in cells as well as lower sensitivity due to the lower number of median UMI counts per cell. The unfixed nuclei were isolated using the above-described NP40-based lysis buffer as described above using the conventional method. The nuclei were cleaned by sorting using the BD FACSMelody™ Cell Sorter available from BD Sciences-US. The sorted nuclei were run using the Single Cell 3′ version 3.1 Reagent Kit available from 10× Genomics. The resulting barcode rank plot shown in FIG. 12 was not as clean and showed more noise. There was no steep drop off and the plot contained multiple “knees” indicating there wasn't a clear separation between the cell-associated barcodes of interest and background. The nuclei also had a much lower fraction of reads in the cells as well as a lower median UMI counts per cell compared to the inventive method, despite the inventive method using a ˜2000 gene target panel vs. the full transcriptome approach of the unbiased gene expression from the unfixed nuclei. Some of the data are shown in Table 13.

TABLE 13
Data for Unfixed Nuclei

| | | | |
|---|---|---|---|
| Number of | 122,502,071 | Estimated | 4,335 |
| reads | | number of | |
| | | cells | |
| Valid barcodes | 96.6% | Fraction reads | 42.9% |
| | | in cells | |
| Valid UMI's | 99.9% | Mean reads | 28,259 |
| | | per cell | |
| Sequencing | 66.5% | Median genes | 611 |
| saturation | | per cell | |
| Q30 bases in | 96.8% | Total genes | 21,900 |
| barcode | | detected | |
| Q30 bases in | 95.3% | Median UMI | 924 |
| RNA read | | counts per | |
| | | cell | |
| Q30 bases in | 92.2% | Q30 bases in | 96.6% |
| sample index | | UMI | |

To further compare the unbiased gene expression approach with the targeted probe approach described in the present invention (the ˜2000 gene target panel), the unbiased gene expression data from the unfixed nuclei was computationally subset from the whole genome to the same ˜2000 gene target panel from the targeted probe panel used. While the targeted reads per cell is lower in this subset gene expression sample compared with the method described in the present disclosure, the complexity achieved by the present method is clearly greater by detecting 299 genes and 1,346 UMIs (Table 11). The subsetted gene expression to the same target gene set yielded 187 genes and 448 UMIs. The fixation methods described herein have clear sample handling benefits over isolating unfixed nuclei and processing using the Single Cell 3′ Kit.

The embodiments of the invention described herein are exemplary, and various modifications and improvements can be made without departing from the spirit and scope of the invention. The scope of the invention is defined by the appended claims, and all changes that fall within the meaning and range of equivalents are intended to be embraced therein.

1-45. (canceled)

46. A method for nucleic acid analysis of tissue samples comprising:
a) contacting a tissue sample with a fixing agent;
b) in the presence of the fixing agent, dividing the tissue sample into tissue segments to allow perfusion of the fixing agent into the tissue segments;
c) dissociating the tissue segments to provide a plurality of biological particles, wherein the biological particles comprise a plurality of sample nucleic acid molecules; and
d) generating a plurality of barcoded nucleic acid molecules using the plurality of sample nucleic acid molecules and a plurality of nucleic acid barcode molecules.

47. The method of claim 46, wherein the tissue sample includes a solid tissue sample, a fresh tissue sample, or a frozen tissue sample.

48. The method of claim 46, wherein the fixing agent includes an organic fixing agent selected from the group consisting of an alcohol, ketone, aldehyde, cross-linking agent, disuccinimidyl suberate (DSS), dimethylsuberimidate (DMS), formalin, dimethyladipimidate (DMA), dithio-bis(-succinimidyl propionate) (DSP), disuccinimidyl tartrate (DST), ethylene glycol bis (succinimidyl succinate) (EGS), bis-imidazole-carboxylate compound, and combinations thereof.

49. The method of claim 46, wherein the fixing agent comprises formalin and includes about 2% to about 6% by weight formaldehyde in water.

50. The method of claim 46, wherein dividing the tissue sample or dissociating the tissue segments includes chemical, enzymatic or mechanical methods.

51. The method of claim 46, wherein subsequent to step (b) or step (c), the method further comprises contacting the tissue segments or biological particles with a quenching medium or quenching solution.

52. The method of claim 46, wherein no decrosslinking or reversible fixation agents are used in the method.

53. The method of claim 46, wherein the plurality of sample nucleic acid molecules includes messenger RNA molecules.

54. The method of claim 46, wherein prior to step (d), the method further comprises hybridizing a plurality of nucleic acid probes to the plurality of sample nucleic acid molecules of the plurality of biological particles.

55. The method of claim 54, wherein a first probe and a second probe hybridize to a sample nucleic acid molecule of a biological particle to form a nucleic acid complex comprising the first probe, the second probe and the sample nucleic acid molecule.

56. The method of claim 55, wherein the nucleic acid complex comprises a barcode sequence.

57. The method of claim 56, wherein the first probe or the second probe comprise the barcode sequence.

58. The method of claim 56, wherein step (d) includes partitioning the plurality of biological particles into a plurality of partitions.

59. The method of claim 58, wherein the plurality of partitions is a plurality of droplets or a plurality of wells.

60. The method of claim 55, wherein the biological particle includes the nucleic acid complex and wherein the biological particle includes a labeling agent.

61. The method of claim 60, wherein the labeling agent is selected from the group consisting of a protein, a peptide, an antibody, a lipophilic moiety, a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a Darpin, and a protein scaffold.

62. The method of claim 61, wherein the labeling agent is configured to couple to a feature of the biological particle, wherein the feature is selected from the group consisting of a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, and adherens junction.

63. The method of claim 60, wherein the labeling agent includes a reporter oligonucleotide.

64. The method of claim 46, wherein the plurality of biological particles includes a plurality of single cells, a plurality of single nuclei, or a plurality of single cells and single nuclei.

65. A composition of a plurality of biological particles from claim 64.
